(19) United States
(12) Patent Application Publication
(10) Pub. No.: US 2007/0059716 A1
(43) Pub. Date: Mar. 15, 2007

(54) Methods for detecting fetal abnormality

(76) Inventors: Ulysses Balis, Peabody, MA (US); Mehmet Toner, Wellesley Hills, MA (US); Ravi Kapur, Stoughton, MA (US); John Walsh, Auburndale, MA (US)

Correspondence Address: WILSON SONSINI GOODRICH & ROSATI, 650 PAGE MILL ROAD, PALO ALTO, CA, 94304-1050, US

(21) Appl. No.: 11/228,454

(22) Filed: Sep. 15, 2005

(51) Int. Cl. C12Q 1/68; G06F 19/00

(52) U.S. Cl. 435/6; 702/20

(57) ABSTRACT

The invention relates to a method of identifying fetal abnormality from a maternal blood sample by capturing an image of a fetal nucleated red blood cell obtained from the maternal blood sample; inputting probe intensities for a plurality of nucleic acid probes that bind fetal nucleic acids of interest; analyzing the probe intensities; and generating a diagnostic output according to results of the analysis. In some embodiments, the probes are specific to a chromosome.

### BACKGROUND OF THE INVENTION

Analysis of specific cells can give insight into a variety of diseases. These analyses can provide non-invasive tests for detection, diagnosis and prognosis of diseases, thereby eliminating the risk of invasive diagnosis. For instance, social developments have resulted in an increased number of prenatal tests. However, the available methods today, amniocentesis and chorionic villus sampling (CVS) are potentially harmful to the mother and to the fetus. The rate of miscarriage for pregnant women undergoing amniocentesis is increased by 0.5-1%, and that figure is slightly higher for CVS. Because of the inherent risks posed by amniocentesis and CVS, these procedures are offered primarily to older women, i.e., those over 35 years of age, who have a statistically greater probability of bearing children with congenital defects. As a result, a pregnant woman at the age of 35 has to balance an average risk of 0.5-1% to induce an abortion by amniocentesis against an age, related probability for trisomy 21 of less than 0.3%.

Some non-invasive methods have already been developed to diagnose specific congenital defects. For example, maternal serum alpha-fetoprotein, and levels of unconjugated estriol and human chorionic gonadotropin can be used to identify a proportion of fetuses with Down's syndrome, however, these tests not one hundred percent accurate. Similarly, ultrasonography is used to determine congenital defects involving neural tube defects and limb abnormalities, but is useful only after fifteen weeks' gestation.

The presence of fetal cells within the blood of pregnant women offers the opportunity to develop a prenatal diagnostic that replaces amniocentesis and thereby eliminates the risk of today's invasive diagnosis. However, fetal cells represent a small number of cells against the background of a large number of maternal cells in the blood which make the analysis time consuming and prone to error.

There are several approaches devised to separate population of cells. These cell separation techniques may be grouped into two categories: (1) methods based on the selection of cells stained using various cell-specific markers, e.g., fluorescence activated cell sorting (FACS) and magnetic activated cell sorting (MACS); and (2) methods for isolation of living cells using a biophysical parameter specific to the population of interest, e.g., charge flow separation. These methods suffer from various limitations such as high cost, low yield, need of skilled operators and in some methods lack of specificity. As a result, no clinically acceptable method for enrichment of rare cell populations, particularly fetal cells, from peripheral blood samples has been devised which yields cell populations sufficient to permit clinical diagnosis. Hence, there is a need for a method for enriching and separating a particular cell type from a mixture that overcomes the limitations of existing technology.

### SUMMARY OF THE INVENTION

In one aspect, the invention relates to methods of identifying fetal abnormality from a maternal blood sample by delivering a maternal blood sample from a pregnant female to an analyzer adapted for capturing an image of one or more fetal cell enriched from said blood sample; analyzing signals from one or more nucleic acid probes that bind fetal nucleic acids; analyzing said signals; and generating a diagnostic output according based on said analyzing step. Such probes can be specific to a chromosome, such as, e.g., X chromosome, Y chromosome, chromosome 21, chromosome 13 and chromosome 18.

In some embodiments, analyzing comprises determining number of said probe signals, determining size of said probe signals, determining shape of said probe signals, determining aspect ratio of said probe signals, or determining distribution of said probe signals. In some embodiments, analyzing comprises capturing an image of a fetal nucleated red blood cell obtained from the matemal blood sample; inputting probe intensities for a plurality of nucleic acid probes that bind fetal nucleic acids of interest; analyzing the probe intensities; and generating a diagnostic output according to results of the analysis. In one embodiment, the probes are specific to a chromosome. In one embodiment, the chromosome is selected from the group consisting of: X chromosome, Y chromosome, chromosome 21, chromosome 13 and chromosome 18. In one embodiment, the analyzing step comprises determining the number of the probes.

In one aspect, the invention relates to a computer program product that detects a condition of a fetus comprising of computer code that detects fetal nucleated red blood cell (fnRBC) in a sample; computer code that receives probe intensities from one or more nucleic acid probes that bind fetal nucleic acids of interest; computer code that analyzes the intensities received; computer code that generates a call according to results of analyzing the probe intensities; and a computer readable medium that stores the computer codes. In one embodiment, the computer readable medium is a memory, hard drive, floppy disk, CD-ROM, flash memory, or tape. In one embodiment, the probes are specific to a chromosome. In one embodiment, the chromosome is selected from the group consisting of: X chromosome, Y chromosome, chromosome 21, chromosome 13 and chromosome 18. In one embodiment, the probes are colorimetric probes. In one embodiment, the probes are fluorescent probes.

### SUMMARY OF THE DRAWINGS

FIG. 1 illustrates one embodiment of a size-based separation module.

FIG. 2 illustrates one embodiment of a size-based separation module with three separate analytes each of a different hydrodynamic size flowing through it.

FIG. 3 illustrates one embodiment of a size-based separation module with bypass obstacles having a cheese wedge shape.

FIG. 4 illustrates one embodiment of a plurality of size-based separation modules in parallel with one another.

FIG. 5 is a table illustrating separation capabilities of one embodiment of the size-based separation module.

FIG. 6 is a picture illustrating cells captured by the capture module.

FIGS. 7A-7C illustrate various embodiments of the capture module.

FIG. 8 illustrates one embodiment of the capture module.

FIGS. 9A-9D illustrate various aspects of the detection module.

FIGS. 10A-B illustrate embodiments of the business methods described herein.

FIGS. 11A-11E illustrate an exemplary size-based separation module of the invention.

FIGS. 12A-F illustrate typical histograms generated by hematology analytes from a blood sample generated by the device.

FIGS. 13A-13D illustrate various embodiments of the size-based separation module.

FIGS. 14A-14D illustrate various embodiments of the size-based separation module.

FIGS. 15A-15B illustrate cell smears of the product and waste fractions.

FIGS. 16A-16D illustrate cell smears of the product and waste fractions.

FIG. 17 illustrates trisomy 21 pathology in an isolated fetal nucleated red blood cell.

FIGS. 18A-18D illustrate an exemplary mask employed to fabricate a size-based separation module.

FIGS. 19A-19G illustrate exemplary SEMs of a size-based separation module.

FIGS. 20A-20D illustrate one embodiment of a mask employed to fabricate a size-based separation module.

FIGS. 21A-21F illustrate exemplary SEMs of a size-based separation module.

FIGS. 22A-22F illustrate exemplary SEMs of a size-based separation module.

FIGS. 23A-23D illustrate mask and portions of a size-based separation module.

FIGS. 24A-24S illustrate exemplary SEMs of a size-based separation module.

FIGS. 25A-25C illustrate an exemplary size-based separation module.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention provides systems, apparatuses, and methods for isolation, separation and enrichment of rare analytes (e.g., organisms, cells, and cellular components) from a sample, a fluid sample, or more preferably a whole blood sample. Table 1 below illustrates examples of various cell types and their concentrations and average sizes in blood in vivo.

TABLE 1
Cell Types, Concentrations, and Sizes of Blood Cells.

| Cell Type | Concentration (cells/μL) | Size (μm) |
|---|---|---|
| Red blood cells (RBC) | 4.2-6.1 × 10⁶ | 4-6 |
| Segmented Neutrophils (WBC) | 3600 | >10 |
| Band Neutrophils (WBC) | 120 | >10 |
| Lymphocytes (WBC) | 1500 | >10 |
| Monocytes (WBC) | 480 | >10 |
| Eosinophils (WBC) | 180 | >10 |
| Basophils (WBC) | 120 | >10 |
| Platelets | 500 × 10³ | 1-2 |
| Fetal Nucleated Red Blood Cells | 2-50 × 10³ | 8-12 |

In some embodiments, the apparatus(es) herein are used for separating or enriching analytes or cell from a fluid mixture wherein said analytes or cells are at a concentration of less than 1×10⁻³, 1×10⁻⁴, 1×10⁻⁵, 1×10⁻⁶, or 1×10⁻⁶ cells/μL of a fluid sample. In some, embodiments, the apparatus(es) herein are used for separating or enriching analytes or cells from a fluid mixture wherein said analytes or cells are at a concentration of less than 1:100, 1:1000, 1:10,000, 1:100,000, 1,000,000, 1:10,000,000 or 1:100,000,000 of all cells in a sample.

In preferred embodiments, the present invention provides systems and apparatuses for separating and enriching one or more cells from a blood sample. For example, fetal cells can be enriched or separated by the systems and methods herein from a maternal blood sample. Also, epithelial, endothelial, progenitor, foam, stem and cancer cells can be enriched from a blood sample. After separation and/or enrichment of these and/or other analytes or rare cells from a fluid sample, the systems herein can be used to detect such analytes and analyze such analytes. Analysis of analytes can be used for various applications as disclosed herein.

I. Sample Collection/Preparation

The systems and methods herein involve obtaining one or more samples from a source to be analyzed. A sample can be obtained from a water source, food, soil, air, animal, etc. If a solid sample is obtained (e.g., tissue sample or soil sample) such solid sample can be liquefied or solubilized prior to subsequent enrichment and/or analysis. If a gas sample is obtained, it may be liquefied or solubilized as well.

In some embodiments, when a sample is derived from an animal, it is preferably derived from a mammal, or more preferably from a human. Examples of fluid samples derived from an animal include, but are not limited to, whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal and genitourinary tracts, and amniotic fluid. Preferably, a fluid sample derived from an animal is a blood sample. When analyzing a fluid sample from an animal, the animal can be, for example, a domesticated animal, such as a cow, a chicken, a pig, a horse, a rabbit, a dog, a cat, and a goat. In preferred embodiments, the animal is a human and the blood sample is a whole blood sample. Blood samples derived from an animal can be used, for example, to screen/diagnose that animal for a condition, or when derived from a pregnant animal to perform prenatal screen. In preferred embodiments, the systems herein contemplate obtaining a blood sample from a pregnant human to screen a fetus for a condition or abnormality.

A fluid sample can be obtained from an animal using any technique known in the art. For example, for drawing blood, a syringe or other vacuum suction device may be used. A fluid sample such as blood is preferably drawn into an evacuated tube or bag.

In some embodiments, a fluid sample obtained from an animal is directly applied to the apparatus(es) herein, while in other embodiments, the sample is pre-treated or processed prior to being delivered to an apparatus of the invention. For example, blood drawn from an animal can be treated with one or more reagents prior to delivery to an apparatus of the invention or it may be collected into a container that is preloaded with such reagent(s). Reagents that are contemplated herein include but are not limited to, a stabilizing reagent, a preservative, a fixant, a lysing reagent, a diluent, an anti-apoptotic reagent, an anti-coagulation reagent, an anti-thrombotic reagent, magnetic property regulating reagents, a buffering reagent, an osmolality regulating reagent, a pH regulating reagent, and/or a cross-linking reagent.

Examples of methods for processing fluid samples and delivering them to an analytical device are described in U.S. Ser. No. 11/071,270, entitled “System For Delivering a Diluted Solution” filed Mar. 3, 2004, and U.S. Ser. No. [Unassigned], entitled “Methods and Systems for Fluid Delivery”, filed Sep. 15, 2005, both of which are incorporated herein by reference for all purposes.

When obtaining a blood sample from an animal, the amount of blood can vary depending upon animal size, its gestation period, condition being screened for, etc. In some embodiments, less than 50 mL, 40 mL, 30 mL, 20 mL, 10 mL, 9 mL, 8 mL, 7 mL, 6 mL, 5 mL, 4 mL, 3 mL, 2 mL, or 1 mL of a fluid sample (e.g., blood) are obtained from the animal. In some embodiments, 1-50 mL, 2-40 mL, 3-30 mL, or 4-20 mL of blood are obtained from an individual. In other embodiments, more than 5, 10, 15, 20, 25, 30, 35, 40, 45, 50, 55, 60, 65, 70, 75, 80, 85, 90, 95 or 100 mL of a fluid sample are obtained from the animal.

An entire sample collected can be applied to the apparatus(es) herein for enrichment and/or separation of rare analytes such as fetal cells and epithelial cells. In some embodiments, samples are obtained at successive time intervals and applied to the apparatus(s) herein for further analysis.

In some embodiments, the systems and methods herein allow enrichment, separation and analysis of rare cells (e.g., fetal cells, epithelial cells, or cancer cells) from a blood sample of less than 10 mL, 5 mL or 3 mL. In some embodiments, the systems and methods herein can be used to enrich rare cells from larger volumes of blood such as those greater than 20 mL, 50 mL, or 100 mL. Any one of the above functions can occur within, for example, less than 1 day, or 12, 10, 11, 9, 8, 7, 6, 5, 4, 3, 2, hours or less than 60, 50, 40, 30, 20, or 10 minutes.

When screening a fetus, a blood sample can be obtained from a pregnant mammal or pregnant human within 24, or more preferably 20, 16, 12, 8, or more preferably 4 weeks of gestation. In other embodiments, screening and detecting fetal cells can occur after pregnancy has terminated.

In some embodiments, a blood sample is combined with a lysate that selectively lyses one or more cells or components in the blood sample, e.g., fetal cells or components of a blood cell. For example, a maternal blood sample comprising fetal cells can be combined with water or another osmolality regulating agent to selectively lyse the fetal cells prior to separation and enrichment of the cellular components of the fetal cells by the systems herein.

Preferably, a blood sample is applied to the system herein within 1 week, 6 days, 5 days, 4 days, 3 days, 2 days, 1 day, 12 hrs, 6 hrs, 3 hrs, 2 hrs, or 1 hr from when the blood is obtained. In some embodiments, a blood sample is applied to a system herein upon withdrawal from an animal. Preferably, the sample is applied to the systems herein at a temperature of 4-37° C.

II. Enrichment

The present invention involves enrichment of rare analytes from a sample. In some embodiments, the rare analytes are cells or cellular components. Examples of rare cells include, but are not limited to, platelets, white blood cells, fetal nucleated red blood cells from maternal blood, epithelial cells, endothelial cells, progenitor cells, cancer cells, tumor cells, bacteria, viruses, protozoan cells and chimera thereof. Examples of cellular components include, but are not limited to, mitochondria, a ribozyme, a lysosome, endoplasmic reticulum, a golgi, a protein, protein complexes and nucleic acids. Such separation is preferably made according to size. A sample of the present invention can be a solid, gaseous, or liquid sample. Solid samples are preferably solubilized or liquefied prior to performing an enrichment step.

Enrichment can be performed using one or more of the methods and apparatuses known in the art, and in particular those disclosed in International Publication Nos. 2004/029221 and 2004/113877, U.S. Publication No. 2004/0144651, U.S. Pat. Nos. 5,641,628, 5,837,115 and 6,692,952, and U.S. Application Nos. 60/703,833, 60/704,067, 60/668,415, 10/778,831, 11/071,679, and 11/146,581, all of which are incorporated herein by reference for all purposes. In preferred embodiments, enrichment or separation of analytes occur using one or more size-based separation modules (e.g., sieves, matrixes, electrophoretic modules); and optionally one or more capture modules (e.g., an affinity-based separation module, antibodies, and magnetic beads).

1. Size-Based Separation

Size based separation modules can separate analyte(s) from a fluidic sample based on the hydrodynamic sizes of analytes in the sample. In preferred embodiments, a size-based separation module comprises one or more two-dimensional arrays of obstacles which form an array of gaps. Arrays of obstacles are preferably two-dimensional and can have obstacles/gaps which are preferably staggered. The arrays are configured such that fluid passing through a gap in an array is divided unequally into subsequent gaps. An angle of deflection can be, for example, at least 10, 20, 30, 40, 50, 60, or 70% of pitch. Preferably, a separation module can be adapted to deflect analytes that are larger than a critical size away from the array of obstacles and into a bypass channel. In some embodiments, a size-based separation module comprises more than 10, 100, 1,000, 10,000 or 100,000 obstacles. When the obstacles are aligned in a two-dimensional array, the array can have, for example, more than 2, 10, 20, 30, 40, 50, 60, 70, 80, 90, 100, 120, 140, 160, 180, 200, 400, 600, 800, or 1000 rows of obstacles.

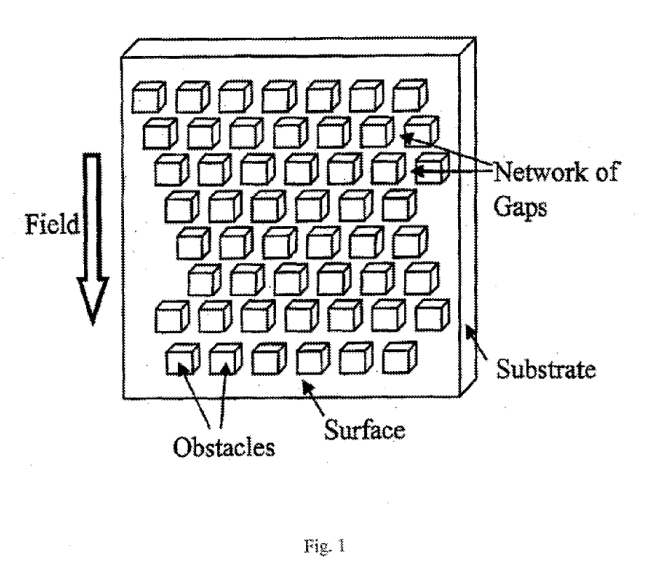

In preferred embodiments, either gaps, obstacles, or both may be of mesoscale (less than 1 mm in one direction). FIG. 1 illustrates an exemplary size-based separation module. Obstacles (which may be of any shape) are coupled to a flat substrate to form an array of gaps. A transparent cover or lid may be used to cover the array. The obstacles form a two-dimensional array with each successive row being staggered from the one above and below. Average fluid flow is designated by the field array. In some embodiments, arrays of obstacles are designed to allow passage and processing of at least 1 mL, 2 mL, 5 mL, 10 mL, 20 mL, 50 mL, 100 mL, 200 mL, or 500 mL of fluid sample per hour. The flow of sample into a size-based separation module can be aligned at a small angle (flow angle) with respect to a line-of-sight of the array. Optionally, a size-based separation module can be coupled to an infusion pump to perfuse the sample through the obstacles.

The size-based separation modules herein can be configured such that analytes (e.g., cells) having a hydrodynamic size larger than a critical size migrate along the line-of-sight in the array, whereas those having a hydrodynamic size smaller than the critical size follow the flow in a different direction. Hydrodynamic size of an analyte depends in part on the analyte's physical dimensions, osmolarity of the fluid medium, and the analyte's shape and deformability.

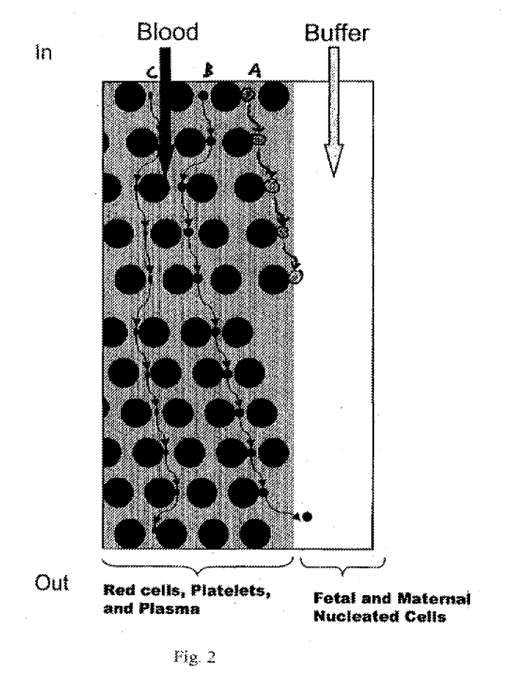

FIG. 2 illustrates this embodiment; a first path A is the deterministic path for a first analyte having a first hydrodynamic size. A second path which is more tortuous within the obstacles is the deterministic path for a second analyte having a hydrodynamic size smaller than said first analyte. The second analyte is seen to flow more in the average flow direction through the array than the first analyte. It follows a deterministic path B. Also, a third analyte, which has a hydrodynamic size smaller than both the first and second analytes, travels in path C, which is exclusively within the array of obstacles and the average fluid path.

Multiplexing

In any of the embodiments herein, one or more arrays obstacles are fluidly coupled in series or in parallel.

In some embodiments more than 1, 2, 3, 4, 5, 6, 7, 8, 9, 10, 15, 20, 25, 30, 35, 40, 45, 50, 60, 70, 80, 90, or 100 separation modules are fluidly coupled in parallel. Preferably about 10-20 of such modules are fluidly coupled in parallel. Fluidly coupling more than one separation module in parallel allows for high-throughput analysis of the sample assayed (e.g., more than 1, 1.5, 2, 2.5, 3, 3.5, 4, 4.5, 5, 10, 15, 20, 30, 40, 50, 60, 70, 80, 90, or 100 mL of a fluid sample per hour, or more preferably more than 5 mL of fluid sample per hour).

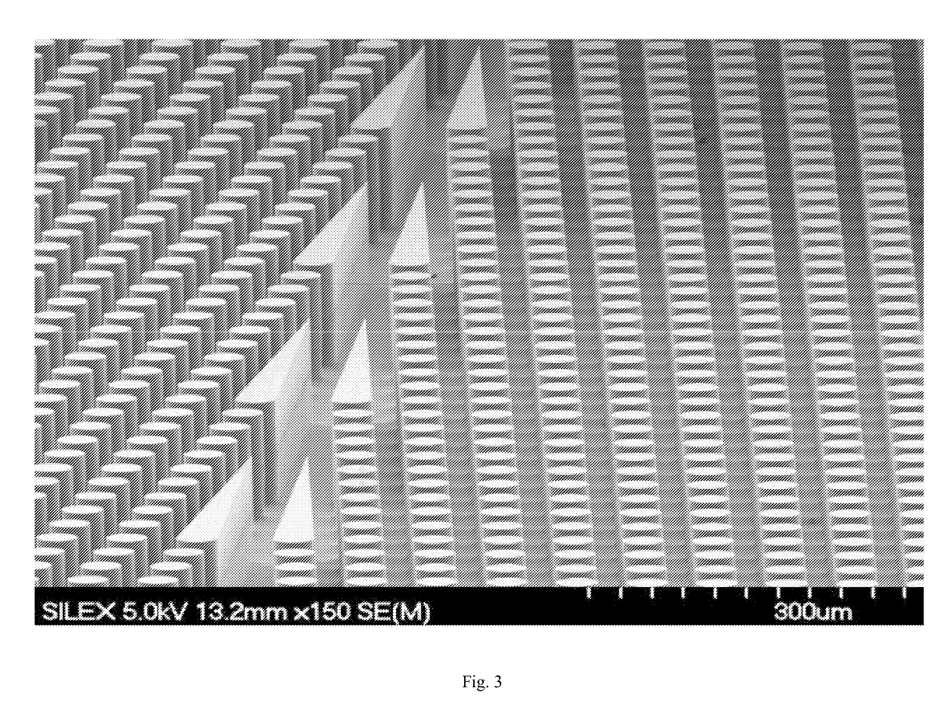

FIG. 3 illustrates one embodiment of multiplexing. In FIG. 3, two arrays of obstacles are disposed side-by-side, e.g., as mirror images. In such arrangement, the critical size of the two arrays may be the same or different. Moreover, the arrays may be arranged such that the major flux flows to the boundary of the two arrays, to the edge of each array, or a combination thereof. Such a duplexed array may also contain a central region disposed between the two arrays to collect particles above the critical size or to alter the sample (e.g., through buffer exchange, reaction, or labeling). In FIG. 3 the central region or bypass channel is disposed within obstacles shaped like cheese wedges to prevent backflow.

Putting multiple arrays on one device in parallel increases sample-processing throughput, and allows for parallel processing of multiple samples or portions of the sample for different fractions or manipulations. It also increases the flow rate of fluid being processed by the separation module. When performing parallel processing of the same sample, outlets may or may not be fluidly connected. For example, when the plurality of arrays has the same critical size, the outlets may be connected for high throughput samples processing. In another example, the arrays may not all have the same critical size or the particles in the arrays may not all be treated in the same manner, and the outlets may not be fluidly connected. In some embodiments, multiplexing is achieved by placing a plurality of duplex arrays on a single device. A plurality of arrays, duplex or single, may be placed in any possible three-dimensional relationship to one another. In some embodiments, a multiplex device comprises two or more arrays of obstacles fluidly coupled in series. For example, an output from the major flux of one device may be coupled to an input of a second device. Alternatively, an output from the minor flux of one device may be coupled to an input of the second device.

In another embodiment, multiple arrays are employed to separate an analyte over a wide size range. For example, a device can have three arrays fluidly coupled in series, but any other number of arrays may be employed. Typically, the cut-off size in the first array (most upstream array) is larger than the cut-off in the second array (adjacent and downstream from the first array), and the first array cut-off size is smaller than the maximum pass-through size of the second array. The same is true for any subsequent array. The first array will deflect (remove) analytes that may clog the second array. Similarly, the second array will deflect (and remove) analytes that may clog the third array.

As described, in a multiple-stage array (multiplexed array), large particles, e.g., cells that could cause clogging downstream, are deflected first, and these deflected particles need to bypass the downstream stages to avoid clogging. Thus, devices of the invention may include bypass channels that remove output from an array. Although described here in terms of removing particles above the critical size, bypass channels may also be employed to remove output from any portion of the array.

In any of the embodiments herein, a separation module preferably has specificity greater than 50%, 60%, 70%, 80%, 90%, 95%, 96%, 97%, 98%, 99%, 99.1%, 99.2%, 99.3%, 99.4%, 99.5%, 99.6%, 99.7%, 99.8%, 99.9% or 99.95% for separating an analyte of interest from a fluid sample (especially a fetal cell or epithelial cell). In any of the embodiments herein, a separation module preferably has sensitivity greater than 50%, 60%, 70%, 80%, 90%, 95%, 96%, 97%, 98%, 99%, 99.1%, 99.2%, 99.3%, 99.4%, 99.5%, 99.6%, 99.7%, 99.8%, 99.9% or 99.95% for separating an analyte of interest from a fluid sample (especially a fetal cell or epithelial cell).

Moreover, in any of the embodiments herein, an analyte of interest can be concentrated from an initial concentration of less than 5, 2, 1, 5×10⁻¹, 2×10⁻¹, 1×10¹, 5×10⁻², 2×10⁻², 1×10⁻², 5×10⁻³, 2×10⁻³, 1×10⁻³, 5×10⁻⁴, 2×10⁻⁴, 1×10⁻⁴, 5×10⁻⁵, 2×10⁻⁵, 1×10⁻⁵, 5×10⁻⁶, 2×10⁻⁶, 1×10⁻⁶¹, 5×10⁻⁷, 2×10⁻⁷, or 1×10⁻⁷ analytes/mL fluid sample. Also, in any of the embodiments herein the separation module can separate an analyte (e.g., cell) that is less than 1% of all analytes in a sample or less than 1%, 0.5%, 0.2%, 0.1%, 0.05%, 0.02%, 0.01%, 0.005%, 0.002%, 0.001%, 0.0005%, 0.0002%, 0.0001%, 0.00005%, 0.00002%, 0.00001%, 0.000005%, 0.000002%, or 0.000001% of all analytes (e.g., cells) in a sample (e.g., a blood sample derived from an animal such as a human). The separation module herein can increase the concentration of such analytes of interest by transferring them from the fluid sample to an enriched sample (sometimes in a new fluid medium, such as a buffer). The new concentration of the analytes in the enriched sample can be at least 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, or 5,000,000,000 fold more concentrated than in the original sample.

Inlets/Outlets

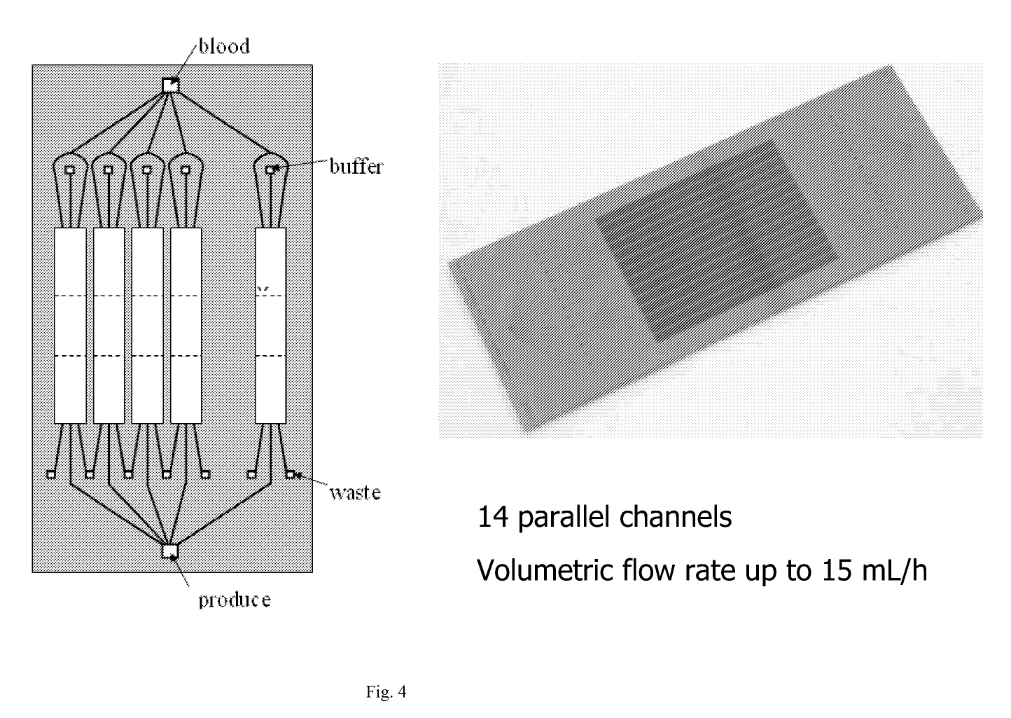

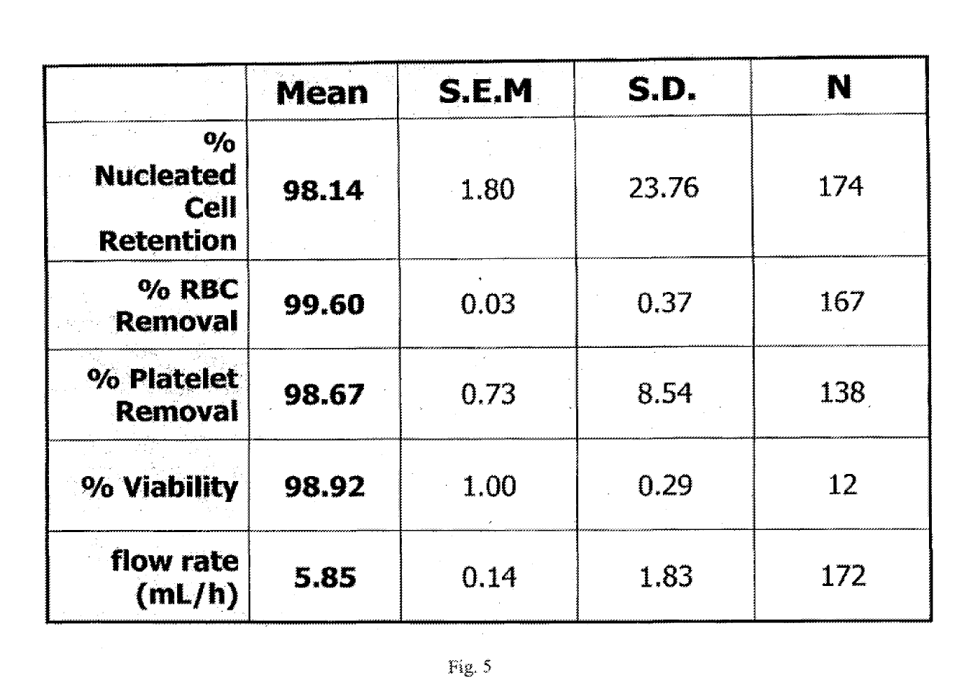

Moreover, the number of inlets and/or outlets may vary depending on the intended use of the device. In a preferred embodiment, a single array of obstacles comprises two or more outlets. An example of such an array is illustrated in FIG. 4 wherein 14 pairs of arrays are disposed as mirror images of one another. Each array thus has a first inlet for delivering a sample and a second inlet for delivering a reagent such as a buffer to the array. Each array also has a first outlet for waste (undesirable products) and a second outlet for product (analytes of interest).

In some embodiments, a size-based separation module includes a first outlet for removal of larger analytes which are directed away from the average direction of flow and a second outlet for removal of smaller analytes, which flow through the array of obstacles in the average direction of flow. Additional outlets can be provided to collect fractions during various points in the separation procedure. Furthermore, in some embodiments, more than one inlet is contemplated for a single two dimensional array. The inlets can provide additional samples and/or reagents, including for example, a stabilizing reagent, a preservative, a fixant, a lysing reagent, a diluent, an anti-apoptotic reagent, labeling reagent, an anti-coagulation reagent, an anti-thrombotic reagent, a buffering reagent, an osmolality-regulating reagent, a pH-regulating reagent, a stabilizer, a PCR reagent, a washing solution, and/or a cross-linking reagent.

In some embodiments, cells of interest (e.g., fetal cells) can be selectively lysed and then a fluid sample comprising the cellular components of the cells of interest can pass over the separation module. Cellular components of interest can be separated from other cells in a blood sample based on size using the methods disclosed herein or known in the art. When a lysing regent is delivered to a separation device simultaneously with a sample, or when a sample is first mixed with a lysing reagent and then delivered to the separation devices herein the device may be configured to deflect/separate one or more cellular organelles such as, for example, a nucleus, a mitochondria, a ribozyme, a lysosome, an endoplasmatic reticulum or a golgi. For example, in some embodiments, a maternal blood sample is mixed with a lysing reagent that selectively lyses fetal nucleated red blood cells. Such lysing reagent can be, for example, water or any other agent known in the art to selectively lyse fetal cells. The blood sample is then delivered to a device herein that selectively deflects all or substantially all other analytes from the blood sample, thus enriching the concentration of organelles (e.g., nuclei) of the fetal red blood cells. In such an embodiment, the nuclei will come out of the “waste” outlet. In other embodiments, the lysing reagent is delivered in a second inlet along with the blood sample. In this embodiment, lysing occurs on the device concurrently with the separation.

In some embodiments, one or more analyte(s) may be contacted with binding moieties (e.g., magnetic beads), that selectively bind the agents and increase their size (hydrodynamic size). Unbound analytes and unbound binding moieties may be removed based on their smaller size (e.g., via the “waste” outlet), while the bound analytes may be deflected and removed based on size from a different outlet.

Device configuration and/or geometry may also be designed in various manners. For example, circular inlets and outlets may be used. (See FIG. 4 as an example of circular inlets.) An entrance region devoid of obstacles is then incorporated into the design to ensure that blood cells are uniformly distributed when they reach the region where the obstacles are located. Similarly, the outlet is designed with an exit region devoid of obstacles to collect the exiting cells uniformly without damage.

Bypass Channel

As the analytes and/or cells of a fluid sample flow through the array of obstacles, those having a hydrodynamic size greater than a critical size will be deflected to a bypass channel. A bypass channel is characterized as having a channel wider than the average gap between obstacles. Moreover, a bypass channel has a width equal to or larger than the largest component (largest cell) separated from the sample. For example, in some embodiments, a bypass channel in a separation module can have a width greater than 50, 60, 70, 80, 90, 100, 110, 120, 130, 140, 150 microns. In some embodiments, a main channel has a width of less than 100, 90, 80, 70, 60, 50, 40, 30, or 20 microns.

A bypass channel can also be characterized by the obstacles that surround it or form its outer edges. Such obstacles are preferably adapted to prevent backflow or turbulence of larger cells or analytes that have reached the bypass channel. In some embodiments, bypass channel obstacles have a straight edge parallel to the main channel and flow direction. In some embodiments, a bypass channel obstacle has a cross section in the shape of a cheese wedge, wherein the pointed end of the wedge is directed downstream. (See FIG. 3)

In some embodiments a single bypass channel is used, and one or more stages (arrays) share the bypass channel. In some embodiments, multiple bypass channels are used. For example, each of a plurality of stages can have its own bypass channel. In one embodiment, larger analytes (e.g., fetal cells, epithelial cells, tumor cells) are deflected into the major flux and then into a bypass channel to prevent clogging. Smaller cells that would not cause clogging proceed to the second stage where they are further separated according to size. This design may be repeated for as many stages as desired. At each stage, the bypass channel can be fluidly connected to an outlet, thus allowing for collection of multiple fractions from a sample. Bypass channels can also be designed to maintain constant flux through a device, remove an amount of flow so the flow in the array is not perturbed, or increase the amount of flow in certain regions. Similarly, portions of the boundaries of arrays may be designed to generate unique flow patterns (e.g., flow-feeding, flow extracting, etc.).

In any of the embodiments herein, each array thus has a maximum pass-through size that is several times larger than the cut-off size. This result may be achieved using a combination of larger gaps and smaller bifurcation ratio ε. In certain embodiments, the ε is at most 1/2, 1/3, 1/10, 1/30, 1/100, 1/300, or 1/1000. Also, in such embodiments, obstacle shape may affect the flow profile in the gap; however, the obstacles can be compressed in the flow direction, in order to make the array short. Single stage arrays may include bypass channels as described herein.

Shape of Obstacles

Dimensions and geometry of obstacles in a size-based separation module may be uniform or may vary to form uniform or non-uniform patterns. For example, obstacles may have cylindrical, moon shape, or square cross sections. In preferred embodiments, obstacles are cylindrical, such that the obstacle has a round cross-section. Obstacles preferably have a diameter (longest cross sectional length) of between 4-40 microns, 5-30 microns, 6-20 microns, or 7-10 microns. In some embodiments, a separation obstacle has a diameter of more than 1, 2, 3, 4, 5, 6, 7, 8, 9, 10, 11, 12, 13, 14, 15, 16, 17, 18, 19, 20, 30, 40, or 50 microns. In some embodiments, a separation obstacle has a diameter of less than 100, 90, 80, 70, 60, 50, 40, 30, 20, or 10 microns. The distance between obstacles may also vary. In some embodiments, the distance between obstacles is at least 10, 25, 50, 75, 100, 250, 500, or 750 μm. In some embodiments, the distance between the obstacles is at most 1000, 750, 500, 250, 100, 75, 50, or 25 μm. Moreover, the diameter, width, or length of the obstacles may be at least 5, 10, 25, 50, 75, 100, or 250 μm and at most 500, 250, 100, 75, 50, 25, or 10 μm. The height of obstacles can also vary but preferably is equal to or greater than the height of the largest analyte being separated. In some embodiments, separation obstacles have a height ranging from 10-500 microns, 20-200 microns, 30-100 microns, or 40-50 microns. In some embodiments, separation obstacles have a height less than 1500, 1000, 500, 400, 300, 200, 100, 90, 80, 70, 60, 50, 40, 30, 20, or 10 microns.

Analyte Sizes

In some embodiments, a separation module has a first separation region adapted to separate an analyte (rare cell) from a fluid sample, wherein the analyte has a hydrodynamic size greater than 20, 19, 18, 17, 16, 15, 14, 13, 12, 11, 10, 9, 8, 7, 6, 5, 4, 3, 2, or 1 micron. More preferably a separation module has a first separation region adapted to separate an analyte from a fluid sample, wherein the analyte has a hydrodynamic size greater than 15, 14, 13, 12, 11, 10, 9, 8, 7, 6, 5, or 4 microns. More preferably a separation module has a first separation region adapted to separate an analyte from a fluid sample, wherein the analyte has a hydrodynamic size greater than 10, 9, 8, 7, or 6 microns.

In one embodiment, a separation module has a first separation region and a second separation region wherein the first separation region is adapted and configured to separate an analyte with a hydrodynamic size of at least 15, 20, 25, 30, 35, or 40 microns or greater, and the second separation region is adapted to separate an analyte with a hydrodynamic size of at least 10, 15, 20, 25, 30, or 35 microns or greater wherein the critical size of the first region is greater than the critical size of the second region. The first and second separation regions can be in fluid communication (fluidly coupled) with one another, such that the second separation region is downstream and in series with the first separation region. In some embodiments, the separation module can also comprise a third separation region adapted to separate components having a hydrodynamic size of at least 5, 10, 15, 20, 25, or 30, microns or greater wherein the critical size of the second region is greater than the critical size of the first region. The third separation region is fluidly coupled to said second separation region and is downstream of it. The separation module can optionally comprise additional regions as described above, each of which separates smaller and smaller components from a sample.

In one embodiment, a separation module is adapted to direct analytes in a sample having a hydrodynamic size (e.g., diameter) of 15 microns or greater in a direction away from the flow direction of smaller components and into a main channel; a second separation region adapted to direct components in a sample having a hydrodynamic size (e.g.; diameter) of 7.5 microns or greater in a direction away from the flow direction of smaller components and into a main channel; and a third separation region adapted to direct components in a sample having a hydrodynamic size (e.g., diameter) of 5 microns or greater in a direction away from the flow direction of smaller components and into a main channel. The above embodiment is especially useful for separating red blood cells from a blood sample.

Of course, the above separation module can be adjusted to separate smaller or larger components from a liquid sample. For example, in some embodiments a separation module can be configured to separate all components that have a dimension greater than 4 microns (e.g., fetal nucleated RBC's, nucleated RBC, and WBC). In some embodiments, a separation module is adapted to separate nucleated cells in a blood sample from non-nucleated cells.

In some embodiments, a separation device can be used to concentrate a cell type or component of interest out of a fluid sample (e.g., a blood sample, urine sample, or other bodily samples) wherein the cell type or component of interest is found in vivo at a concentration of less than 50, 40, 30, 20, or 10% of all blood cells, or more preferably less than 9, 8, 7, 6, 5, 4, 3, 2, or 1% of all blood cells, or more preferably less than 0.9, 0.8, 0.7, 0.6, 0.5, 0.4, 0.3, 0.2, or 0.1% of all blood cells, or more preferably less than 0.09, 0.08, 0.07, 0.06, 0.05, 0.04, 0.03, 0.02, or 0.01% of all blood cells, or more preferably less than 0.009, 0.008, 0.007, 0.006, 0.005, 0.004, 0.003, 0.002, or 0.001% of all blood cells, or more preferably less than 0.0009, 0.0008, 0.0007, 0.0006, 0.0005, 0.0004, 0.0003, 0.0002, or 0.0001% of all blood cells, or more preferably less than 0.00009, 0.00008, 0.00007, 0.00006, 0.00005, 0.00004, 0.00003, 0.00002, or 0.00001% of all cells or components.

Specificity/Sensitivity

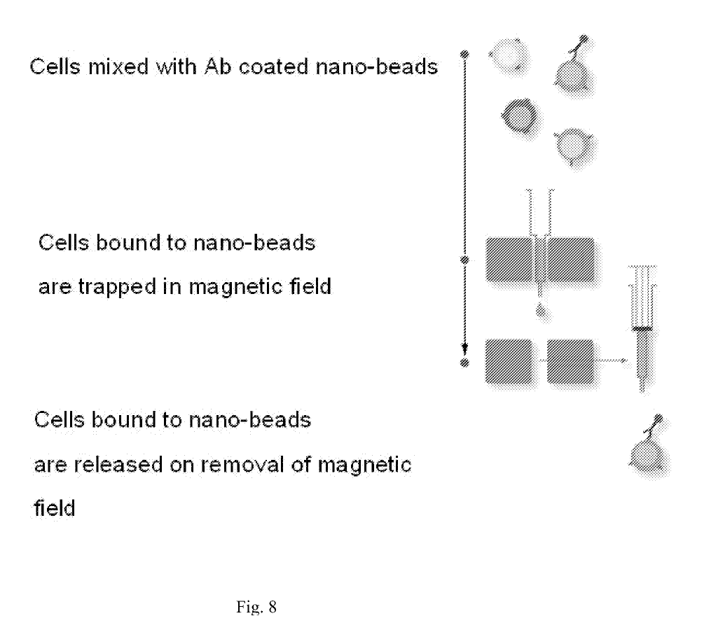

In any of the embodiments herein a size-based separation device can be used for separating one or more cell types from a mixed cell population (e.g., whole blood) with increased efficiency. For example, a size-based separation device preferably retains after separation ≧50%, ≧60%, ≧70%, ≧80%, ≧90%, ≧91%, ≧92%, ≧93%, ≧94%, ≧95%, ≧96%, ≧97%, ≧98%, ≧99%, ≧99.9% of all nucleated cells from a whole blood sample, or more preferably more than ≧50%, ≧60%, ≧70%, ≧80%, 90%, ≧91%, ≧92%, ≧93%, ≧94%, ≧95%, ≧96%, ≧97%, ≧98%, 99%, ≧99.9% of all nucleated fetal red blood cells from a maternal blood sample. Similarly, the above devices can retain after separation ≧50%, ≧60%, ≧70%, ≧80%, ≧90%, ≧91%, ≧92%, 93%, ≧94%, ≧95%, ≧96%, ≧97%, ≧98%, 99%, ≧99.9% of all epithelial cells from a blood sample or ≧50%, ≧60%, ≧70%, ≧80%, 90%, ≧91%, ≧92%, ≧93%, ≧94%, ≧95%, ≧96%, ≧97%, ≧98%, ≧99%, ≧99.9% of all cancer cells from a blood sample. Simultaneously, the separation module herein can also remove ≧95%, ≧96%, ≧97%, ≧98%, ≧99%, ≧99.9% of all unwanted analytes (e.g., red blood cells and platelets) from a fluid sample, such as for example whole blood. FIG. 8 illustrates some examples of specificity and sensitivity achieved by one embodiment of the size-based separation modules herein.

Any or all of the above steps can occur with minimal dilution of the product. In some embodiments, desired analytes of interest are retained and separated into a solution that is less than 50, 40, 30, 20, 10, 9.0, 8.0, 7.0, 6.0, 5.0, 4.5, 4.0, 3.5, 3.0, 2.5, 2.0, 1.5, 1.0, or 0.5 fold diluted from the original sample. In some embodiments, any or all of the above steps occur while the desired product is concentrated. For example, enriched analytes of interest may be at least 1.5, 2.0, 2.5, 3.0, 3.5, 4.0, 4.5, 5.0, 5.5, 6.0, 6.5, 7.0, 7.5, 8.0, 8.5, 9.0, 10, 20, 30, 40, 50, 60, 70, 80, 90, 200, 300, 400, 500, 600, 700, 800, 900, 1000, 2000, 3000, 4000, 5000, 6000, 7000, 8000, 9000, 10,000, 100,000, 500,000 or 1,000,000 fold more concentrated in the final enriched solution than in the original sample. For example, a 10 times concentration increase of a first cell type out of a blood sample means that the ratio of first cell type/all cells in a sample is 10 times greater after the sample was applied to the apparatus herein. Such concentration can take a fluid sample (e.g., a blood sample) of greater than 10 mL or 20 mL total volume comprising rare components of interest, and it can concentrate such rare component of interest into a concentrated solution of less than 5 mL total volume.

In one embodiment, reagents are added to a sample, to selectively or non-selectively increase the hydrodynamic size of analytes within the sample. This modified sample is then delivered through an obstacle array of the present invention. Because the analytes are swollen and have an increased hydrodynamic size, it will be possible to use obstacle arrays with larger and more easily manufactured gap sizes. In a preferred embodiment, the steps of swelling and size-based enrichment are performed in an integrated fashion on a device. Suitable reagents include any hypotonic solution, e.g., de-ionized water, 2% sugar solution, or neat non-aqueous solvents. Other reagents include beads, e.g., magnetic or polymer, that bind selectively (e.g., through antibodies or avidin-biotin) or non-selectively.

In another embodiment, reagents are added to the sample to selectively or non-selectively decrease the hydrodynamic size of the analytes within the sample. A non-uniform decrease in particle size in a sample will increase the difference in hydrodynamic size between analytes. For example, nucleated cells are separated from enucleated cells by hypertonically shrinking the cells. The enucleated cells can shrink to a very small particle, while the nucleated cells cannot shrink below the size of the nucleus. Exemplary shrinking reagents include hypertonic solutions.

In an alternative embodiment, affinity functionalized beads are used to increase the volume of particles of interest relative to the other particles present in a sample, thereby allowing for the operation of an obstacle array with a larger and more easily manufactured gap size.

In any of the embodiments herein, fluids may be driven through a device either actively or passively. Fluids may be pumped using electric field, a centrifugal field, pressure-driven fluid flow, an electro-osmotic flow, and capillary action. In preferred embodiments, the average direction of the field will be parallel to the walls of the channel that contains the array.

1. Separation by Capture

The systems herein can optionally include one or more capture modules. A capture module enriches an analyte (e.g., cell) of interest from a fluid sample by restricting or inhibiting its migration or movement or by complexing it with capture moiety. In some embodiments, the capture module utilizes affinity based separation though affinity based separation is only optional.

A capture module herein is highly specific and selective. In any of the embodiments herein, a capture module preferably has specificity greater than 50%, 60%, 70%, 80%, 90%, 95%, 96%, 97%, 98%, 99%, 99.1%, 99.2%, 99.3%, 99.4%, 99.5%, 99.6%, 99.7%, 99.8%, 99.9% or 99.95% for separating an analyte of interest (e.g., a fetal cell or epithelial cell) from a fluid sample. In any of the embodiments herein, a capture module preferably has sensitivity greater than 50%, 60%, 70%, 80%, 90%, 95%, 96%, 97%, 98%, 99%, 99.1%, 99.2%, 99.3%, 99.4%, 99.5%, 99.6%, 99.7%, 99.8%, 99.9% or 99.95% for separating an analyte of interest (e.g., a fetal cell or epithelial cell) from a fluid sample.

Moreover, in any of the embodiments herein, an analyte of interest can be separated (e.g., concentrated) by a capture module from an initial concentration of less than 5, 2, 1, 5×10⁻¹, 2×10¹, 1×10⁻¹, 5×10⁻², 2×10⁻², 1×10⁻², 5×10⁻³, 2×10⁻³, 1×10⁻³, 5×10⁻⁴, 2×10⁻⁴, 1×10⁻⁴, 5×10⁻⁵, 2×10⁻⁵, 1×10⁻⁵, 5×10⁻⁶, 2×10⁻⁶, 1×10⁻⁶¹, 5×10⁻⁷, 2×10⁻⁷, or 1×10⁻⁷ analytes/μL fluid sample. Also, in any of the embodiments herein a capture module can separate an analyte (e.g., cell) that is less than 1% of all analytes in a sample or less than 1%, 0.5%, 0.2%, 0.1%, 0.05%, 0.02%, 0.01%, 0.005%, 0.002%, 0.001%, 0.0005%, 0.0002%, 0.0001%, 0.00005%, 0.00002%, 0.00001%, 0.000005%, 0.000002%, or 0.000001% of all analytes (e.g., cells) in a sample (e.g., a blood sample derived from an animal such as a human). A capture module can increase the concentration of such analytes of interest by at least 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, or 5,000,000,000 fold of their original sample concentrations.

In some embodiments, a capture module comprises a channel with an array of obstacles. The obstacles can be of one or more shapes. The array is preferably two-dimensional, and the obstacles can be uniform or non-uniform in their order. In preferred embodiments, the array comprises a two-dimensional uniform array of staggered obstacles.

Examples of capture modules are disclosed in International Publication No. 2004/029221 and U.S. Pat. Nos. 5,641,628, 5,837,115 and 6,692,952, which are incorporated herein by reference for all purposes.

Shape and Size

It may be desirable to increase the surface area of the obstacles or time of contact between the sample and obstacles in order to increase the amount of binding. Thus, capture obstacles of the present invention can have various shapes and forms to increase their surface area and/or contact time with a sample. Moreover, shape and size of obstacles can vary depending on the analyte being captured, sample concentration etc. The larger the analyte being captured by the capture module, the higher the capture obstacles will be. In some embodiments, the height of an obstacle is less than 1500, 1400, 1300, 1200, 1100, 1000, 900, 800, 700, 600, 500, 400, 300, 200, or 100 microns. In some embodiments, the height of an obstacle is more than 20, 30, 40, 50, 60, 70, 80, 90, 100, 200, 300, 400, or 500 microns.

Similarly, the size of the gap between obstacles will vary depending on the size of obstacle that is being captured. In some embodiments, the gap between obstacles is less than 50, 40, 30, 20, or 10 microns. In some embodiments, the gap between obstacles is less than 10, 9, 8, 7, 6, 5, 4, 3, or 2 fold the hydrodynamic size of the analyte of interest. In some embodiments, the gap between obstacles is less than the hydrodynamic size of the analyte(s) of interest. In such an embodiment, analytes of interests are trapped between obstacles. The present invention contemplates arrays having gaps both wider than the analyte(s) of interest and narrower than the analytes of interest. In some embodiments, restricted gaps (those having a width equal to or less than an analyte of interest) are dispersed either uniformly or non-uniformly throughout the array of obstacles. Preferably, a restricted gap is uniformally dispersed throughout an array of obstacles.

In some embodiments, the diameter of each obstacle is less than 1500, 1400, 1300, 1200, 1100, 1000, 900, 800, 700, 600, 500, 400, 300, 200, 100, 90, 80, 70, 60, 50, 40, 30, or 20 microns. In other embodiments, the diameter of each obstacle is more than 5, 10, 20, 30, 40, 50, 60, 70, 80, 90, or 100 microns.

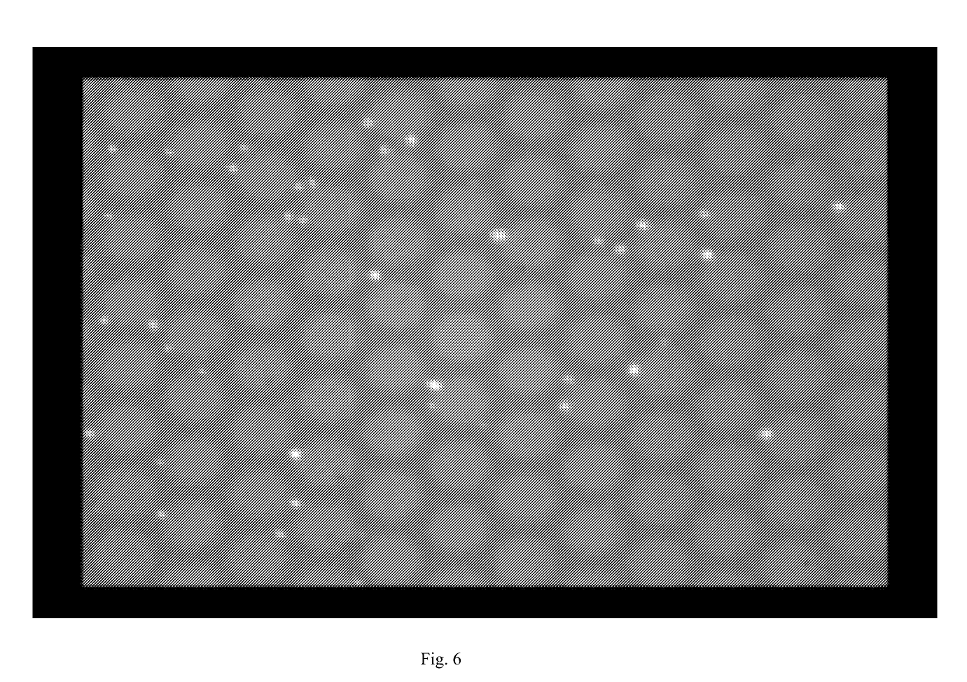

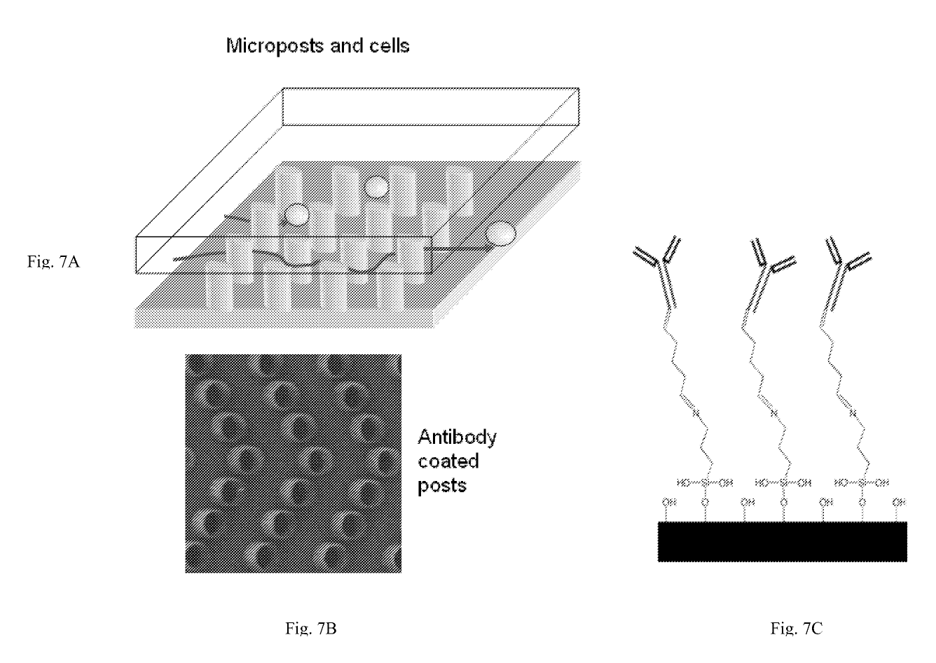

In some embodiments, obstacles in a capture array are adapted to selectively (and optionally reversibly) bind one or more component of a fluid sample either reversibly or non-reversibly. An obstacle can include, for example, one or more capture moieties having an affinity for selected cell(s) or component(s) in a fluid sample. Such capture moiety can comprise an antibody that can specifically bind a cell or component of interest, e.g., fetal cells, red blood cells, white blood cells, platelets, epithelial cells, cancer cells, endothelial cells, or other rare cells. For example, in some embodiments, a capture moiety comprises of an antibody (or fragment thereof) that specifically binds red blood cells or epithelial cells. Such antibodies include, for example anti-CD71 and anti-EpCAM, respectively. In preferred embodiments, such antibodies are monoclonal. Other antibodies that can be included in capture moieties include, but are not limited to, anti-CD235a, anti-CD36, anti-selectins, anti-carbohydrates, anti-CD45, anti-GPA, and anti-antigen i. FIG. 6 illustrates an embodiment of the present invention wherein fetal cells are bound to obstacles coupled with a binding moiety (anti-CD71). FIG. 7A illustrates a path of a first analyte through an array of posts wherein an analyte that does not specifically bind to a post continues to migrate through the array, while an array that does bind a post is captured by the array. FIG. 7B is a picture of antibody coated posts. FIG. 7C illustrates coupling of antibodies to a substrate (e.g., obstacles, side walls, etc.) as contemplated by the present invention.

As with the separation module, a capture module can have multiple regions, each of which selectively binds different cell(s) and/or component(s) of interest. A system comprising a multi-region capture module will include two or more capture regions fluidly coupled to one another in series. Moreover, a system can comprise a plurality of separation modules fluidly coupled in parallel to increase the amount of sample being simultaneously analyzed.

When enriching a first cell type from a mixed cell population (e.g., blood), preferably, at least 60%, 70%, 80%, 90%, 95%, 98%, or 99% of cells that are capable of binding to the surfaces of the capture module are removed from the mixture. The surface coating of the capture module is preferably designed to minimize nonspecific binding of cells. For example, at least 99%, 98%, 95%, 90%, 80%, or 70% of cells or analytes not capable of binding to the binding moiety are not bound to the surfaces of the capture module. The selective binding in the capture module results in the separation of a specific analyte (e.g., living cell population) from a mixture of cells. Obstacles are present in the device to increase surface area for analytes (e.g., cells) to interact with while in the chamber containing the obstacles so that the likelihood of binding is increased. The flow conditions are such that analyte cells are very gently handled in the device without the need to deform mechanically in order to go in between the obstacles. Positive pressure or negative pressure pumping or flow from a column of fluid may be employed to transport cells into and out of the microfluidic devices of the invention (e.g., capture modules).

Preferably, the methods herein retain at least 50%, 60%, 70%; 80%, 90%, 95%, 96%, 97%, 98%, 99%, 99.1%, 99.2%, 99.3%, 99.4%, 99.5%, 99.6%, 99.7%, 99.8%, 99.9% or 99.95% of the desired analytes (e.g., cells) compared to the initial mixture, while potentially concentrating the population of desired analytes by a factor of at least 100, 1000, 10,000, 100,000, or 1,000,000 relative to the amount of analytes in a sample.

In some embodiments, a capture module comprises more than 10, 100, 1,000, 10,000 or 100,000 obstacles. When such obstacles are aligned in a two-dimensional array, the array can have, for example, more than 2, 10, 20, 30, 40, 50, 60, 70, 80, 90, 100, 120, 140, 160, 180, 200, 400, 600, 800, or 1000 rows of obstacles.

Magnetic

In some embodiments, the capture module involves the use of magnetic particles, magnetic fields, and/or magnetic devices/components of devices for purposes of separating and/or enriching one or more analytes.

Magnetic particles of the present invention can come in any size and/or shape. In some embodiments, a magnetic particle has a diameter of less than 500 nm, 400 nm, 300 nm, 200 nm, 100 nm, 90 mm, 80 nm, 70 nm, 60 nm or 50 nm. In some embodiments, a magnetic particle has a diameter that is between 10-1000 nm, 20-800 nm, 30-600 nm, 40-400 nm, or 50-200 nm. In some embodiments, a magnetic particle has a diameter of more than 10 nm, 50 nm, 100 nm, 200 nm, 500 nm, 1000 nm, or 5000 nm. The magnetic particles can be dry or suspended in a liquid. Mixing of a fluid sample with a second liquid medium containing magnetic particles can occur by any means known in the art including those described in U.S. Ser. No. [Not Assigned], entitled “Methods and Systems for Fluid Delivery,” filed Sep. 15, 2005.

In some embodiments, when an analyte in a sample (e.g., analyte of interest or not of interest) is ferromagnetic or otherwise has a magnetic property, such analyte can be separated or removed from one or more other analytes (e.g., analyte of interest or not of interest) or from a sample depleted of analytes using a magnetic field. FIG. 8 illustrates an embodiment of this capture mechanism wherein a first analyte is coupled to antibodies that specifically bind the first analyte and wherein the antibodies are also coupled to nano-beads. When a mixture of analytes comprising the first analyte-nanobead complex and a second analyte are delivered into a magnetic field, the first analyte-nanobead complex will be captured while other cells continue to migrate through the field. The first analyte can then be released by removing the magnetic field.

The magnetic field can be external or internal to the devices herein. An external magnetic field is one whose source is outside a device herein (e.g., container, channel, obstacles) contemplated herein. An internal magnetic field is one whose source is within a device contemplated herein.

In some embodiments, when an analyte desired to be separated (e.g., analyte of interest or not of interest) is not ferromagnetic or does not have a magnetic property, a magnetic particle can be coupled to a binding moiety that selectively binds such analyte. Examples of binding moieties include, but are not limited to, polypeptides, antibodies, nucleic acids, etc. In preferred embodiments, a binding moiety is an antibody that selectively binds to an analyte of interest (such as a red blood cell, a cancer cell, or an epithelial cell). Therefore, in some embodiments a magnetic particle may be decorated with an antibody (preferably a monoclonal antibody) selected from the group consisting of: anti-CD71, anti-CD45, anti-EpiCAM, or any other antibody disclosed herein.

Magnetic particles may be coupled to any one or more of the devices herein prior to contact with a sample or may be mixed with the sample prior to delivery of the sample to the device(s).

In some embodiments, the systems herein include a reservoir containing a reagent (e.g., magnetic particles) capable of altering a magnetic property of the analytes captured or not captured. The reservoir is preferably fluidly coupled to one or more of the devices/modules herein. For example, in some embodiments, a magnetic reservoir is coupled to a size-based separation module and in other embodiments a magnetic reservoir is coupled to a capture module.

The exact nature of the reagent will depend on the nature of the analyte. Exemplary reagents include agents that oxidize or reduce transition metals, reagents that oxidize or reduce hemoglobin, magnetic beads capable of binding to the analytes, or reagents that are capable of chelating, oxidizing, or otherwise binding iron, or other magnetic materials or particles. The reagent may act to alter the magnetic properties of an analyte to enable or increase its attraction to a magnetic field, to enable or increase its repulsion to a magnetic field, or to eliminate a magnetic property such that the analyte is unaffected by a magnetic field.

Any magnetic particles that respond to a magnetic field may be employed in the devices and methods of the invention. Desirable particles are those that have surface chemistry that can be chemically or physically modified, e.g., by chemical reaction, physical adsorption, entanglement, or electrostatic interaction.

Capture moieties can be bound to magnetic particles by any means known in the art. Examples include chemical reaction, physical adsorption, entanglement, or electrostatic interaction. The capture moiety bound to a magnetic particle will depend on the nature of the analyte targeted. Examples of capture moieties include, without limitation, proteins (such as antibodies, avidin, and cell-surface receptors), charged or uncharged polymers (such as polypeptides, nucleic acids, and synthetic polymers), hydrophobic or hydrophilic polymers, small molecules (such as biotin, receptor ligands, and chelating agents), carbohydrates, and ions. Such capture moieties can be used to specifically bind cells (e.g., bacterial, pathogenic, fetal cells, fetal blood cells, cancer cells, and blood cells), organelles (e.g., nuclei), viruses, peptides, proteins, carbohydrates, polymers, nucleic acids, supramolecular complexes, other biological molecules (e.g., organic or inorganic molecules), small molecules, ions, or combinations (chimera) or fragments thereof. Specific examples of capture moieties for use with fetal cells include anti-CD71, anti-CD36, anti-selectins, anti-GPA, anti-carbohydrates, and holotransferrin. Thus, in another embodiment, the capture moiety is fetal cell specific.

Once a magnetic property of an analyte has been altered, it may be used to effect an isolation or enrichment of the analyte relative to other constituents of a sample. The isolation or enrichment may include positive selection by using a magnetic field to attract the desired analytes to a magnetic field, or it may employ negative selection to attract an analyte not of interest. In either case, the population of analytes containing the desired analytes may be collected for analysis or further processing.

The device used to perform the magnetic separation may be any device that can produce a magnetic field (e.g., any of the devices or reservoirs described herein). In one embodiment, a MACS column is used to effect separation of the magnetically altered analyte. If the analyte is rendered magnetically responsive by the reagent (e.g., using any reagent described herein), it may bind to the MACS column, thereby permitting enrichment of the desired analyte relative to other constituents of the sample.

In another embodiment, separation may be achieved using a device, preferably a microfluidic device, which contains a plurality of magnetic obstacles. If an analyte in the sample is modified to be magnetically responsive (e.g., through a reagent that enhances an intrinsic magnetic property of the analyte or by binding of a magnetically responsive particle to the analyte), the analyte may bind to the obstacles, thereby permitting enrichment of the bound analyte. Alternatively, negative selection may be employed. In this example, the desired analyte may be rendered magnetically unresponsive, or an undesired analyte may be bound to a magnetically responsive particle. In this case, an undesired analyte or analytes will be retained on the obstacles whereas the desired analyte will not, thus enriching the sample for the desired analyte.

Magnetic regions of the device can be fabricated with either hard or soft magnetic materials, such as, but not limited to, rare earth materials, neodymium-iron-boron, ferrous-chromium-cobalt, nickel-ferrous, cobalt-platinum, and strontium ferrite. Portions of the device may be fabricated directly out of magnetic materials, or the magnetic materials may be applied to another material. The use of hard magnetic materials can simplify the design of a device because they are capable of generating a magnetic field without other actuation. Soft magnetic materials, however, enable release and downstream processing of bound analytes simply by demagnetizing the material. Depending on the magnetic material, the application process can include cathodic sputtering, sintering, electrolytic deposition, or thin-film coating of composites of polymer binder-magnetic powder. A preferred embodiment is a thin film coating of micromachined obstacles (e.g., silicon posts) by spin casting with a polymer composite, such as polyimide-strontium ferrite (the polyimide serves as the binder, and the strontium ferrite as the magnetic filler). After coating, the polymer magnetic coating is cured to achieve stable mechanical properties. After curing, the device is briefly exposed to an external induction field, which governs the preferred direction of permanent magnetism in the device. The magnetic flux density and intrinsic coercivity of the magnetic fields from the posts can be controlled by the % volume of the magnetic filler.

In another embodiment, an electrically conductive material is micropatterned on the outer surface of an enclosed microfluidic device. The pattern may consist of a single, electrical circuit with a spatial periodicity of approximately 100 microns. By controlling the layout of this electrical circuit and the magnitude of the electrical current that passes through the circuit, one can develop periodic regions of higher and lower magnetic strength within the enclosed microfluidic device.

The magnetic particles can be disposed uniformly throughout a device or in spatially resolved regions. In addition, magnetic particles may be used to create structure within the device. For example, two magnetic regions on opposite sides of a channel can be used to attract magnetic particles to form a “bridge” linking the two regions.

As described, the invention features analytical devices for the enrichment of analytes such as bacteria, viruses, fungi, cells, cellular components, viruses, nucleic acids, proteins, protein complexes, carbohydrates, and fragments or combination (chimera) thereof. In addition to altering a magnetic property, the devices may be used to effect various manipulations on analytes in a sample. Such manipulations include enrichment or concentration of a particle, including size-based fractionization, or alteration of the particle itself or the fluid carrying the particle. Preferably, the devices are employed to enrich rare analytes (rare cells) from a heterogeneous mixture or to alter a rare analytes, e.g., by exchanging the liquid in the suspension or by contacting an analyte with a reagent. Such devices allow for a high degree of enrichment with limited stress on cells, e.g., reduced mechanical lysis or intracellular activation of cells.

Although primarily described in terms of cells, the devices of the invention may be employed with any analytes whose size allows for separation in a device of the invention.

Devices of the invention may be employed in concentrated samples, e.g., where analytes are touching, hydrodynamically interacting with each other, or exerting an effect on the flow distribution around another analyte. For example, the method can separate white blood cells from red blood cells in whole blood from a human donor. Human blood typically contains ˜45% of cells by volume. Cells are in physical contact and/or coupled to each other hydrodynamically when they flow through the array.

The methods of the invention may involve separating from a sample one or more analytes based on a magnetic property of the one or more analytes. In one embodiment, the sample is treated with a reagent that alters a magnetic property of the analyte. The alteration may be mediated by a magnetic particle. In one example, the particle (e.g., a magnetic particle) may be bound to a surface of the device, and desired analytes (e.g., rare cells such as fetal cells, pathogenic cells, cancer cells, or bacterial cells) in a sample may be retained in the device. Thus, the analyte or analytes of interest may then bind to the surfaces of the device. In another embodiment, desired analytes are retained in the device through size-, shape- or deformability-based mechanisms. In another embodiment, negative selection is employed, where the desired analyte is not bound by the magnetic particles. Any of the embodiments may use a MACS column for retention of an analyte (e.g., an analyte bound to a magnetic particle). In the case of positive selection, it is desirable that at least 60%, 70%, 80%, 90%, 95%, 98%, or 99% of the analytes are retained in the device. The surfaces of the device are desirably designed to minimize nonspecific binding of non-target analytes. For example, at least 99%, 98%, 95%, 90%, 80%, or 70% of non-target analytes are not retained in the device. The selective retention in the device can result in the separation of a specific analyte population from a mixture, e.g., blood, sputum, urine, and soil, air, or water samples.

The selective retention of analytes is obtained by introduction of magnetic particles into a device of the invention. Capture moieties may be bound to the magnetic particles to affect specific binding of the target analyte. In another embodiment, the magnetic particles may be disposed such as to only allow analytes of a selected size, shape, or deformability to pass through the device. Combinations of these embodiments are also envisioned. For example, a device may be configured to retain certain analytes based on size and others based on binding. In addition, a device may be designed to bind more than one analyte of interest, e.g., in a serial, parallel, or interspersed arrangement of regions within the device or where two or more capture moieties are disposed on the same magnetic particle or on adjacent particles, e.g., bound to the same obstacle or region. Further, multiple capture moieties that are specific for the same analytes (e.g., anti-CD71 and anti-CD36) may be employed in the device, either on the same or different magnetic particles, e.g., disposed on the same or different obstacle or region.

Magnetic particles may be attached to obstacles present in the device (or manipulated to create obstacles) to increase surface area for analytes to interact with to increase the likelihood of binding. The flow conditions are typically such that the analytes are very gently handled in the device to prevent damage. Positive pressure or negative pressure pumping or flow from a column of fluid may be employed to transport analytes into and out of the microfluidic devices of the invention. The device enables gentle processing, while maximizing the collision frequency of each analyte with one or more of the magnetic particles. The target analytes interact with any capture moieties on collision with the magnetic particles. The capture moieties can be co-localized with obstacles as a designed consequence of the magnetic field attraction in the device. This interaction leads to capture and retention of the target analytes in defined locations. Alternatively, analytes are retained based on an inability to pass through the device, e.g., based on size, shape, or deformability. Captured analytes can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will. In other embodiments, the analytes can be released by disrupting the bond between the analyte and the capture moiety, e.g., through chemical cleavage or interruption of a noncovalent interaction. For example, some ferrous particles are linked to a monoclonal antibody via a DNA linker; the use of DNAse can cleave and release the analytes from the ferrous particle. Alternatively, an antibody fragmenting protease (e.g., papain) can be used to engineer selective release. Increasing the sheer forces on the magnetic particles can also be used to release magnetic particles from magnetic regions, especially hard magnetic regions. In other embodiments, the captured analytes are not released and can be analyzed or further manipulated while retained.

In one embodiment a device is configured to capture and isolate cells expressing the transferrin receptor from a complex mixture. Monoclonal antibodies to CD71 receptor are readily available off-the-shelf covalently coupled to magnetic materials, such as, but not limited to ferrous doped polystyrene and ferroparticles or ferro-colloids (e.g., from Miltenyi and Dynal). The mAB to CD71 bound to magnetic particles is flowed into the device. The antibody coated particles are drawn to the obstacles (e.g., posts), floor, and walls and are retained by the strength of the magnetic field interaction between the particles and the magnetic field. The particles between the obstacles and those loosely retained with the sphere of influence of the local magnetic fields away from the obstacles are removed by a rinse (the flow rate can be adjusted such that the hydrodynamic shear stress on the analytes away from the obstacles is larger than the magnetic field strength).

In addition to the above embodiments, the device can be used for isolation and detection of blood borne pathogens, bacterial and viral loads, airborne pathogens solubilized in aqueous medium, pathogen detection in food industry, and environmental sampling for chemical and biological hazards. Additionally, the magnetic particles can be co-localized with a capture moiety and a candidate drug compound. Capture of a cell of interest can further be analyzed for the interaction of the captured cell with the immobilized drug compound. The device can thus be used to both isolate sub-populations of cells from a complex mixture and assay their reactivity with candidate drug compounds for use in the pharmaceutical drug discovery process for high throughput and secondary cell-based screening of candidate compounds. In other embodiments, receptor-ligand interaction studies for drug discovery can be accomplished in the device by localizing the capture moiety, i.e., the receptor, on a magnetic particle, and flowing in a complex mixture of candidate ligands (or agonists or antagonists). The ligand of interest is captured, and the binding event can be detected, e.g., by secondary staining with a fluorescent probe. This embodiment enables rapid identification of the absence or presence of known ligands from complex mixtures extracted from tissues or cell digests or identification of candidate drug compounds.

Capture Coupled with Size-Based Separation

In the embodiments herein, a size-based separation module(s) and capture module(s) are preferably fluidly coupled. For example a first outlet from a separation module can be fluidly coupled to a capture module. The average flow rate for a sample through the capture module can be the same or different than that in the separation module. In some embodiments, the average flow rate of a sample through the capture module is more than 1, 2, 3, 4, 5, 10, 15, 20, 30, 40, 50, 60, 70, 80, 90, or 100 mL/hour.

In some embodiments, the separation module and capture module are integrated such that a plurality of obstacles acts both to deflect certain analytes according to size and direct them in a path different than the direction of analyte(s) of interest, and also as a capture module to capture, retain, or bind certain analytes based on size, affinity, magnetism or other physical property.

III. Detection/Analysis

In any of the embodiments herein, detection and/or analysis of enriched analytes (e.g., rare cells) or components thereof (e.g., nuclei or chromosomes) can be performed in whole or in part by a person or an analyzer. When enriched analytes are cells, the cells may be permeablized or lysed prior to detection/analysis. An analyzer of the present invention can be automated for high-throughput detection/analysis of enriched analytes (e.g., rare cells from blood or biohazardous analytes). Detection and analysis by an analyzer can occur in sequential steps or can be combined into one step. Preferably, detection and analysis occur in a single step.

An analyzer can include any sample analyzing device known in the art, such as, for example a microscope, a microarray, cell counter, etc. An analyzer can further include one or more computers, databases, memory systems, and system outputs (e.g., a computer screen or printer). In preferred embodiments, an analyzer comprises a computer readable medium, e.g., floppy diskettes, CD-ROMs, hard drives, flash memory, tape, or other digital storage medium, with a program code comprising a set of instructions for detection or analysis to be performed on the enriched analytes. In some embodiments, computer executable logic or program code of an analyzer is stored in a storage medium, loaded into and/or executed by a computer, or transmitted over some transmission medium, such as over electrical wiring or cabling, through fiber optics, or via electromagnetic radiation. When implemented on a general-purpose microprocessor, the computer executable logic configures the microprocessor to create specific logic circuits. Preferably, the computer executable logic performs some or all of the tasks described herein including sample preparation, enrichment, detection and/or analysis.

In some embodiments, an analyzer is fluidly coupled to a size-based separation module or a capture module. In some embodiments, enriched analytes (e.g., cells of interest) are removed from the capture module/size-based separation module and are delivered to a glass slide or cell sorting apparatus for analysis. In preferred embodiments, a cell sorting apparatus allows maintaining a plurality of analytes (e.g., cells) each at an addressable site. Examples of such embodiments are disclosed in U.S. Pat. No. 6,692,952, which is incorporated herein by reference for all purposes. Such module can also include an actuator adapted to selectively release a cell from the addressable site.

In some embodiments, an analyzer is configured to perform a detection step such as visualizing one or more analytes of interest. Visualization of analytes of interest can occur through a transparent cover or lid which covers obstacles in the size-based separation module and/or capture module. In some embodiments, an analyzer comprises a microscope, e.g., as a light microscope, bright field light microscope, fluorescence microscope, electron microscope, etc. (preferably fluidly coupled to a capture module). In some embodiments, an analyzer has dual scanning capabilities (e.g., using a light microscope and a fluorescence microscope). Preferably, an analyzer provides a three-dimensional image of enriched analytes (including analytes of interest). For example, a computer code can detect all nucleated red blood cells, including fetal nucleated red blood cells in an enriched sample. In some embodiments, an analyzer comprises an imaging device such as a camera or video camera. Such imaging device can be used to, capture an image of analytes (including analytes of interest). For example, an imaging device can capture an image of one or more fnRBC obtained from a maternal blood sample. Any of the above may be controllable by computer executable logic that images and saves images of enriched analytes.

In some embodiments, an analyzer is configured to perform an analysis step such as enumerating analytes of interest, e.g., cancer cells, endothelial cell, epithelial cells, etc. Such analyzer can include, for example, a cell counter. The number of analytes of interest detected in a sample can be used by the analyzer or user for making a diagnosis or prognosis of a condition, e.g., cancer). In some embodiments, an analyzer compares (and optionally stores) data collected with known data points. In some embodiments, an analyzer compares (and optionally stores) data collected from case samples and control samples and performs an association study.

In some embodiments, an analyzer comprises a computer executable logic that detects probe signal from one or more probes that selectively bind enriched analytes, analytes of interest, or components thereof. In some embodiments, the computer executable logic also analyzes such signals for their intensity, size, shape, aspect ratio, and/or distribution. The computer executable logic can then general a call based on results of analyzing the probe signals.

Examples of probes whose signals can be detected/analyzed by an analyzer include, but are not limited to, fluorescence probes (e.g., for staining chromosomes such as X, Y, 13, 18 and 21 in fetal cells), chromogenic probes, indirect immunoagents (e.g., unlabeled primary antibodies coupled to secondary enzymes), quantum dots, or other probes that emit a photon. In some embodiments, an analyzer herein detects chromagenic probes, which can provide a significantly faster read time than fluorescent probes. In some embodiments, an analyzer comprises a computer executable logic that performs karyotyping, in situ hybridization (ISH) (e.g., florescence in situ hybridization (FISH), chromogenic in situ hybridization (CISH), nanogold in situ hybridization (NISH)), restriction fragment length polymorphism (RFLP) analysis, polymerase chain reaction (PCR) techniques, flow cytometry, electron microscopy, quantum dots, and nucleic acid arrays for detection of single nucleotide polymorphisms (SNPs) or levels of RNA. In some embodiments, two or more probes are used. For example, multiple FISH probes or other DNA probes may be used in analyzing a single cell or component of interest. Methods for using FISH to detect rare cells are disclosed in Zhen, D. K., et al. (1999) Prenatal Diagnosis, 18(11), 1181-1185, Cheung, M C., (1996) Nature Genetics 14, 264-268, which are incorporated herein by reference for all purposes. Methods for using CISH are disclosed in Arnould, L. et al British Journal of Cancer (2003) 88, 1587-1591; and US Application Publication No. 2002/0019001, which are incorporated herein by reference for all purposes.

For example, when analyzing fetal cells enriched from maternal blood, an analyzer is configured to detect fetal cells or components thereof. In some embodiments, analysis of fetal cells or components thereof is used to determine the sex of a fetus; the presence/absence of a genetic abnormality (e.g., chromosomal/DNA/RNA abnormality); or one or more SNPs. Examples of autosomal abnormalities that can be detected by an analyzer herein include, but are not limited to, Angleman syndrome (15q11.2-q13), cri-du-chat syndrome (5p-), DiGeorge syndrome and Velo-cardiofacial syndrome (22q11.2), Miller-Dieker syndrome (17p13.3), Prader-Willi syndrome (15q11.2-q13), retinoblastoma (13q14), Smith-Magenis syndrome (17p11.2), trisomy 13, trisomy 16, trisomy 18, trisomy 21 (Down's syndrome), triploidy, Williams syndrome (7q11.23), and Wolf-Hirschhorn syndrome (4p-). Examples of sex chromosome abnormalities that can be detected by an analyzer herein include, but are not limited to, Kallman syndrome (Xp22.3), steroid sulfate deficiency (STS) (Xp22.3), X-linked ichthiosis (Xp22.3), Klinefelter syndrome (XXY); fragile X syndrome; Turner syndrome; metafemales or trisomy X; monosomy X, etc. Other less common chromosomal abnormalities that can be detected/analyzed by the analyzers herein include, but are not limited to, deletions (small missing sections); microdeletions (a minute amount of missing material that may include only a single gene); translocations (a section of a chromosome is attached to another chromosome); and inversions (a section of chromosome is snipped out and reinserted upside down).

In some embodiments, an analyzer detects analytes (e.g., cells) stained for an antigen selected from the group consisting of γ and ε globins, Glycophorin A (GPA), i-antigen, and CD35. In particular, an analyzer herein can detect cells stained with anti-ε or anti-γ globin antibodies, or a combination thereof. A combination of γ and ε globins has been found on 95-100% of fNRBC from 10-24 weeks gestation. Al Mufti et al., (2001) Haematologica 85, 357-362; Choolani et al., (2003) Mol. Hum. Reprod. 9, 227-235. The ε-γ combination, or γ globin alone, has been shown to stain fNRBC. See Bohmer, (1998); Choolani et al., (2003); Christensen et al., (2005) Fetal Diagn. Ther. 20, 106-112; and Hennerbichler et al., (2002) Cytometry, 48, 87-92. Antibodies to both globins are known to those skilled in the art and can be obtained from various vendors. Staining can result in a binary score such as positive or negative or in various intensities indicating amount of antigen in the analytes.

In some embodiments, an analyzer detects analytes (e.g., cells) stained for GPA and/or CD71. GPA is present throughout the red blood cell lineage. Thus, it can be used for identifying nucleated red blood cells, regardless of their level of maturation. GPA is thought to be found exclusively on erythroid lineage cells, and is generally found on very few circulating cells, and its presence increases during pregnancy. FACS sorting has shown a combination of CD71 and GPA to be present on at least 0.15% of mononucleated cells during pregnancy. Price et al., (1991) Am. J. Obstet Gynecol., 165, 1713-1717; Sohda et al., (1997) Prenat. Diagn., 17, 743-752. In some embodiments, an analyzer is configured to detect probes specific to CD71 and GPA.

In some embodiments, an analyzer detects analytes (e.g., cells) stained for antigen-i. The i-antigens were first described in the 1950s using patient polyclonal sera. Subsequent data demonstrated that the two forms of the antigen, “I” or “i”, were expressed on adult and fetal cells respectively. More recent structural evidence has defined these antigens as linear and branched repeats of N-acetyllactosamine. The “i” structure arises from the action of two enzymes, β-1,3-N-acetyleglucosaminyltransferase and β-1,4-galactosyltransferase. Conversion of the “i” antigen to the “I” occurs via the enzyme, (β-1,6-N-acetyleglucosaminyltransferase. The genes and chromosomal loci for these enzymes have recently been identified. Yu et al., (2001) Blood, 98, 3840-3845. And more recently, antibodies for the i-antigens have been generated. Antibodies to antigen-i have been used in early work in the field on fetal cells. Kan et al., (1974) Blood 43, 411-415. They have also been recently used for screens of fetal cells obtained by differential density centrifugation. Sitar et al., (2005) Exp. Cell. Res., 302, 153-161. Thus, antibodies and antibody fragments that specifically bind antigen-i can be used for by the methods and compositions herein to enrich, separate, and detect fetal cells. Additionally, the i antigen identifies a greater number of fetal cells in a maternal blood sample (Sitar et al) and provides improvements in the speed of reading results.

In some embodiments, an analyzer comprises a computer executable logic or computer program code that provides a set of instructions identifying/characterizing rare analytes, such as rare cells, in an enriched sample. The code can further provide instruction for imaging such rare analytes and storing such images. In one example, the computer executable logic directs a microscope to identify rare cells (e.g., fetal cells or epithelial cells). The code can further provide a set of instructions for identifying a probe that selectively binds such rare cells or components thereof, e.g., an antibody that specifically binds to ε globin, γ globin, fetal hemoglobin, GPA, i-antigen, CD71, EpCAM, or a combination thereof.

For example, in some embodiments, a computer executable logic provides instructions to identify fetal nucleated red blood cells in a sample; identify and enumerate components of rare cells such as chromosomes; detect probes that specifically bind chromosome 13, 18, 21, X and/or Y; detect one or more single nucleotide polymorphisms (SNPs), detect mutations in genetic sequence; detect levels of mRNA; detect levels of microRNA; etc. The computer executable logic can also include code that detects and/or compares probe intensities e.g., from one or more nucleic acid probes that bind fetal nucleic acids of interest (e.g., chromosomes X, Y, 13, 18, or 21); and code that generates a call according to results of analyzing the probe intensities.

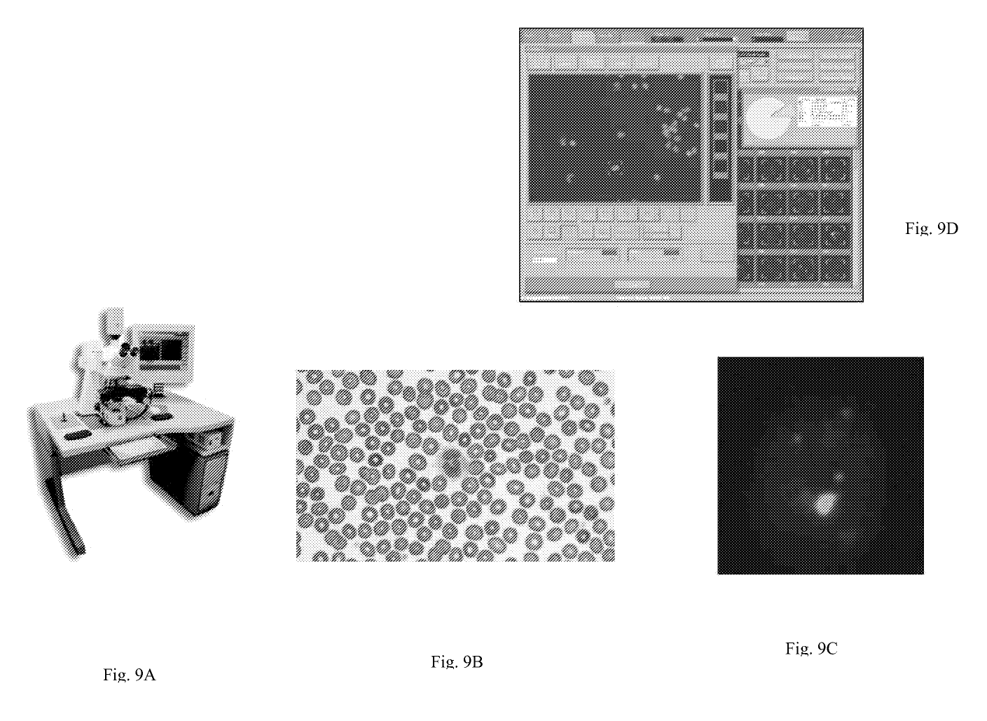

FIGS. 9A-D illustrate an embodiment of the present invention. FIG. 9A illustrates a computer coupled to a microscope which is coupled to a slide or cell arraying module. The microscope analyzes the cells on the slide or cell array. FIG. 9B illustrates cells as visualized by a bright field microscope. FIG. 9C illustrates an XXY cell. FIG. 9D illustrates an image of cells in a field of vision. It also illustrates various features of the code herein to detect various levels of intensities of probes.

In any of the embodiments, an analyzer comprises computer executable logic that controls flow rate of a sample through one or more of the various modules herein.

IV. Applications

The devices/modules and methods herein can be used for various applications including, but not limited to, those already disclosed.

a. Prenatal Diagnosis

In some embodiments, the systems and methods herein can be used to perform a prenatal diagnosis. For example, a peripheral blood sample from a pregnant animal (preferably a human) can be obtained and enriched using one or more of the methods and devices, which are disclosed herein. Preferably, the maternal blood sample is first enriched using one or more size-based modules to separate analytes in the blood sample that have a hydrodynamic size greater than 4 microns (e.g., fetal nucleated red blood cells and maternal white blood cells) from other analytes (e.g., enucleated red blood cells and platelets). Subsequently, the enriched sample comprising the fetal nucleated red blood cells and maternal white blood cells is further separated using one or more capture modules. Preferably, the capture modules positively select (selectively and reversibly bind) the fetal blood cells over the white blood cells. Such capture modules preferably do not use magnetic particles. In some embodiments, a capture module comprises one or more arrays of obstacles covered with anti-CD71 monoclonal antibody. Cell that are captured by such device are then subjected to genetic analysis using one or more FISH assay, PCR amplification, RNA analysis, DNA analysis, etc. In some embodiments, FISH assays are used to detect the presence or absence of aneuploidy. In some embodiments, DNA or RNA analysis is used to detect one or more SNPs or or mRNA levels in the enriched fetal cells. An analyzer comprising computer executable logic that detect sand analyzes fetal cells can be used to automate the system. The analyzer can further comprise a microscope or a microarray.

b. Cancer Diagnosis

In some embodiments, the systems and methods herein can be used to perform a cancer diagnosis. For example, a peripheral blood sample or other fluid sample can be obtained from an animal suspected or known for having cancer. The sample can then be flowed through one or more size-based modules to separate analytes from the sample analytes that have a hydrodynamic size greater than 8, 10, 12, 14, 16, 18, or 20 microns. In some embodiments, enriched cells are one or more cells selected from the group consisting of: an infected WBC, a stem cell, a progenitor cell, an epithelial cell, an endothelial cell, an endometrial cell, a tumor cell, and a cancer cell. In some embodiments, the enriched analytes are optionally flowed through one or more capture modules as described herein.

Enriched cells can then be analyzed to determine, e.g., the number of epithelial cells in the sample, the number of endothelial cells in the sample, the ratio of epithelial/endothelial in the sample, the profile of all cells greater than the critical size, the migration pattern of all cells greater than the critical size, and the change in such characteristics based on at least a second sample obtained from the same animal at a different point in time.

In some embodiments, analysis can involve applying the enriched cells into one or more capture modules that selectively capture cells in a particular size range or that selectively bind cells of interest (e.g., cancer cells expressing one or more cancer markers on their surface or epithelial cells). In some embodiments, enriched cells are further analyzed to determine the presence or absence of an intracellular cancer maker. Any of the embodiments herein can further involve the use of an analyzer to detect, enumerate, and analyze the cells.

Neoplastic conditions whose diagnosis or prognosis is contemplated by the present invention include those selected from the group consisting of: breast cancer, skin cancer, bone cancer, prostate cancer, liver cancer, lung cancer, brain cancer, larynx cancer, gallbladder cancer, pancreas cancer, rectum cancer, parathyroid cancer, thyroid cancer, adrenal cancer, neural tissue cancer, head cancer, neck cancer, colon cancer, stomach cancer, bronchi cancer, kidney cancer, basal cell carcinoma, squamous cell carcinoma, metastatic skin carcinoma, osteo sarcoma, Ewing's sarcoma, veticulum cell sarcoma, myeloma, giant cell tumor, small-cell lung tumor, gallstone tumor, islet cell tumor, primary brain tumor, acute and chronic lymphocyctic and granulocytic tumors, hairy-cell tumor, adenoma, hyperplasia, medullary carcinoma, pheochromocytoma, mucosal neuromas, interstinal ganglioneuromas hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythemia vera, adenocarcinoma, glioblastoma multiforma, acute myeloid leukemia, acute promyelocytic leukemia, acute lymphoblastic leukemia, chronic myelogenous leukemia, myelodysplastic syndrome, lymphomas, malignant melanomas, and epidermoid carcinomas.

c. Veterinary Diagnosis

In some embodiments, the systems and methods herein can be used to perform a veterinary diagnosis. A veterinary diagnosis can involve obtaining a fluid sample (e.g., a blood sample) from an animal, which is preferably domesticated. Examples of domesticated animals include, but are not limited to, a cow, a chicken, a pig, a horse, a rabbit, a dog, a cat, and a dog, a cat, a fish, and a goat. The sample is then enriched using one or more size-based modules to separate analytes from the sample analytes that have a unique hydrodynamic size, e.g., greater than 4, 6, 8, 10, 12, 14, 16, 18, or 20 microns or a hydrodynamic size range (e.g., 6-12 microns or 8-10 microns, etc.). The enriched analytes may be optionally subjected to one or more additional enrichment steps prior to their analysis. For example, in some embodiments, the enriched analytes are optionally flowed through one or more capture modules as described herein.

In some embodiments, analytes enriched from a sample are fetal cells. Such cells can then be analyzed to determine sex of a fetus or a condition in the fetus.

In some embodiments, analytes enriched from a sample are pathogens. Examples of pathogens that can be enriched from the animal include, but are not limited to bacteria, viruses, and protozoa. (Of course such applications are not limited to domesticated animals and also apply to humans.) Once enriched, the cells are analyzed using a detection/analyzer as contemplated herein. Such analyzer can perform gram positive tests, viral load test, FISH assay, PCR assays, etc. to determine to type of pathogen infection, its source, a therapy treatment, etc.

In some embodiments, analytes enriched from a sample are epithelial cells or circulating cancer cells. Such cells can be further analyzed to determine the origin of a cancer affecting the animal, severity of the condition, effectiveness of a therapy treatment, etc.

d. Biodefense

In some embodiments, the systems and methods herein can be used as biodefense or detect the presence of biohazardous material (e.g., a biohazardous analyte). Biohazardous analytes include, but are not limited to, organisms that are infectious to humans, animals or plants (e.g. parasites, viruses, bacteria, fungi, prions, rickettsia); cellular components (e.g., recombinant DNA); and biologically active agents (e.g., toxins, allergens, venoms) that may cause disease in other living organisms or cause significant impact to the environment or community. Examples of pathogens that can be biohazardous analytes include those selected from the group consisting of: Yersinia pestis, Bacillus anthracis, Clostridium botulinum Francisella tularensis, Coxiella burnetii, Brucella spp., Burkholderia mallei, Burkholderia pseudomallei, Streptococcus, Ebola virus, Lassa virus, SARS, Variola major, Alphaviruses, Rickettsia prowazekii, Chlamydia psittaci, Salmonella spp., Escherichia coli 0157.H7, Vibrio cholerae, Cryptosporidium parvum, Nipah virus, hantavirus, as well as chimera of any of the above. Biohazardous material can be detected using the methods and systems herein in, for example, a food sample, a water sample, an air sample, a soil sample, or a biological sample from an animal or plant.

In some embodiments, a sample analyzed by the methods and systems herein can have biohazardous analytes that are less than 1%, 0.1%, 0.01%, 0.001%, 0.0001%, 0.00001%, or 0.000001%, of all analytes in the sample. Moreover, in any of the embodiments, a biohazardous analyte can be at an initial concentration of less than 5, 2, 1, 5×10⁻¹, 2×10⁻¹, 1×10⁻¹, 5×10⁻², 2×10⁻², 1×10⁻², 5×10⁻³, 2×10⁻³, 1×10⁻³, 5×10⁻⁴, 2×10⁻⁴, 1×10⁻⁴, 5×10⁻⁵, 2×10⁻⁵, 1×10⁻⁵, 5×10⁻⁶, 2×10⁻⁶, 1×10⁻⁶, 5×10⁻⁷, 2×10⁻⁷, or 1×10⁻⁷ biohazardous analytes/mL fluid sample. When analyzing a non-fluid sample, the sample is preferably solubilized or liquefied by any means known in the art.

The sample analyzed for biohazardous material is flowed through one or more of the size-based separation modules herein. Preferably, such size-based separation module increases the concentration of the biohazardous analyte by at least 1,000 or 10,000 fold. Enriched analytes can be also optionally flowed through one or more of the capture modules described herein.

After enrichment, the biohazardous analyte are further analyzed using an analyzer. The analyzer optionally comprises a microscope, a microarray, a cell counter, reagents for performing a Gram test, reagents for performing a viral load analysis (e.g., PCR reagents), etc.

e. Research

The systems and methods herein can further be utilized for performing research. For example, in some embodiments, the systems and methods herein are used to perform association studies based on data collected from a plurality of control samples and a plurality of case samples. For example, fluid samples (e.g., blood samples) can be collected from more than 10, 20, 50, or 100 case individuals (individuals with a phenotypic condition) and from more than 10, 20, 50, or 100 control individuals (those not inhibiting the phenotypic condition). Samples from each individual can then be enriched for a first or a plurality of analytes. Such analytes are then enumerated and/or characterized. Data from the above steps is collected and subsequently used to perform an association study. Data is preferably stored in an electronic database. The association study can be performed using a computer executable logic for identifying one or more characteristics associated with case or control samples.

In preferred embodiments, fluid samples obtained from individuals for an association study are blood samples. In preferred embodiments, the analytes enriched from such samples are ones that have a hydrodynamic size greater than 4 microns, or greater than 6, 8, 10, 12, 14, or 16 microns. In some embodiments, samples obtained from individuals are enriched for one or more cells selected from the group consisting of: a RBC, a fetal RBC, a trophoblast, a fetal fibroblast, a white blood cell (WBCs), an infected WBC, a stem cell, an epithelial cell, an endothelial cell, an endometrial cell, a progenitor cell, a cancer cell, a viral cell, a bacterial cell, and a protozoan. Preferably, cells that are enriched are those that are found in vivo at a concentration of less than 1×10⁻¹, 1×10⁻², or 1×10⁻³ cells/mL. Preferably, at least 99% of the cells of interest (those enriched) from the sample are retained. Enrichment for purposes of conducting an association study can increase the concentration of a first cell type of interest by at least 10,000 fold.

The enriched analytes are then analyzed to determine one or more characteristics. Such characteristics can include, e.g., the presence or absence of the analyte in the sample, quantity of an analyte, ratio of two analytes (e.g., endothelial cells and epithelial cells), morphology of one or more analytes, genotype of analyte, proteome of analyte, RNA composition of analyte, gene expression within an analyte, microRNA levels, or other characteristic traits of the analytes enriched are subsequently used to perform an association study.

When a characteristic is associated with the control samples, such characteristic can subsequently be used as a diagnostic for the absence of the phenotypic condition in a patient being tested. When a characteristic is associated with the case samples, it can subsequently be used as a diagnostic for the presence of the phenotypic condition in a patient being tested.

Examples of phenotypic conditions that are contemplated by the present invention, include but are not limited to cancer, endometriosis, infection (e.g., HIV, bacterial, etc.), inflammation, ischemia, trauma, fetal abnormality, etc.

V. Kits

The present invention contemplates kits for enriching analytes from a fluid sample.

In some embodiments, such kits can include, for example, one or more separation module, optionally coupled to capture module(s) adapted to enrich fetal cells from a maternal blood sample.

Separation modules preferably have sensitivity and sensitivity greater than 98% or greater than 99% for enriching the fetal cells. In some embodiments, one or more capture modules are fluidly coupled to the separation module(s). Preferably both separation and capture modules are on the same substrate. The kits herein can further include a set of instructions for analyzing the enriched fetal cells for making a prenatal diagnosis. Examples of prenatal diagnoses that can be made by the kits herein include, but are not limited to, sex of a fetus, existence of trisomy 13, trisomy 18, trisomy 21 (Down Syndrome), Turner Syndrome (damaged X chromosome), Klinefelter Syndrome (XXY) or another irregular number of sex or autosomal chromosomes, or a condition selected from the group consisting of: Wolf-Hirschhorn (4p-), Cri-du-chat (5p-), Williams syndrome (7q11.23), Prader-Willi syndrome (15q11.2-q13), Angelman syndrome (15q11.2-q13), Miller-Dieker syndrome (17p13.3), Smith-Magenis syndrome (17p11.2), DiGeorge and Velo-cardio-facial syndromes (22q11.2), Kallman syndrome (Xp22.3), Steroid Sulfatase Deficiency (STS) (Xp22.3), X-Linked Ichthiosis (Xp22.3), and Retinoblastoma (13q14).

In some embodiments, a kit herein comprises one or more separation module, optionally coupled to capture module(s) adapted to enrich epithelial cells or cancer cells from a blood sample. Such modules preferably have sensitivity and specificity greater than 98% or greater than 99%. Preferably both separation and capture modules are on the same substrate. The kits herein can further include one or more labeling reagents for detection of cancer origin, cancer metastasis, effectiveness of treatment, prognosis, etc. Such reagents can comprise an antibody that specifically binds a cell surface cancer marker. The kits herein can further include a set of instructions for analyzing the enriched fetal cells for making a cancer diagnosis.

Examples of cancers that can be diagnosed using the methods herein include, but are not limited to, breast cancer, skin cancer, bone cancer, prostate cancer, liver cancer, lung cancer, brain cancer, larynx cancer, gallbladder cancer, pancreas cancer, rectum cancer, parathyroid cancer, thyroid cancer, adrenal cancer, neural tissue cancer, head cancer, neck cancer, colon cancer, stomach cancer, bronchi cancer, kidney cancer, basal cell carcinoma, squamous cell carcinoma, metastatic skin carcinoma, osteo sarcoma, Ewing's sarcoma, veticulum cell sarcoma, myeloma, giant cell tumor, small-cell lung tumor, gallstone tumor, islet cell tumor, primary brain tumor, acute and chronic lymphocyctic and granulocytic tumors, hairy-cell tumor, adenoma, hyperplasia, medullary carcinoma, pheochromocytoma, mucosal neuromas, interstinal ganglioneuromas hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythemia vera, adenocarcinoma, glioblastoma multiforma, acute myeloid leukemia, acute promyelocytic leukemia, acute lymphoblastic leukemia, chronic myelogenous leukemia, myelodysplastic syndrome, lymphomas, malignant melanomas, and epidermoid carcinomas.

VI. Business Methods

The systems and methods herein can be used to perform diagnostic services and/or sell diagnostic products. A diagnostic product can include, for example, one or more size-based separation modules, one or more capture modules, a detector, an analyzer, or a combination thereof.

Diagnostic Services—Prenatal

In some embodiments, the business methods herein contemplate providing a prenatal screening service. Such business contemplates obtaining a blood sample from a mammal whose fetus is to be diagnosed. In some embodiments, the business can either draw blood from a patient (animal) that is pregnant or receive a blood sample derived from the pregnant patient. The business herein enriches fetal cells from the blood sample and performs one or more screening test on the fetal cells to determine a condition of the fetus. Examples of conditions that can be determined include, but are not limited to, sex of the fetus, genetic abnormalities such as trisomy 13, 18, 21, X or Y, conditions associated with known SNPs, Wolf-Hirschhorn (4p-), Cri-du-chat (5p-), Williams syndrome (7q11.23), Prader-Willi syndrome (15q11.2-q13), Angelman syndrome (15q11.2-q13), Miller-Dieker syndrome (17p13.3), Smith-Magenis syndrome (17p11.2), DiGeorge and Velo-cardio-facial syndromes (22q11.2), Kallman syndrome (Xp22.3), Steroid Sulfatase Deficiency (STS) (Xp22.3), X-Linked Ichthiosis (Xp22.3), and Retinoblastoma (13q14). All other genetic conditions are also contemplated by the present invention.

The business method then provides a report on the condition in exchange for a service fee. The report can be either provided directly to the patient being tested, a health care provider or insurance company of the patient, or the government.

In some embodiments, the business licenses a CLIA laboratory to perform the enrichment and analysis step. In other embodiments, the business performs the enrichment step and licenses a third party (e.g., a CLIA lab) to perform the analysis step (e.g., genetic testing).

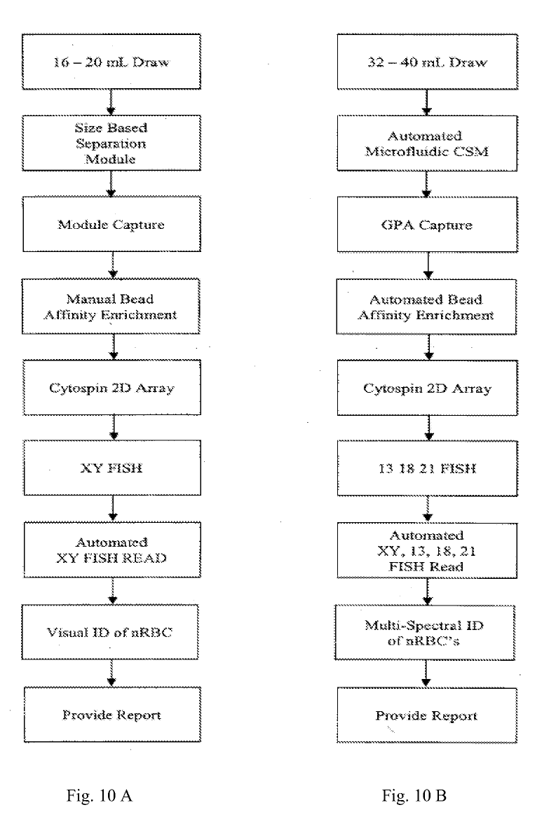

FIG. 10A illustrates one example of the business methods disclosed herein. A blood sample (e.g., 16-20 mL of blood) is drawn from a pregnant woman either by the business herein, the CLIA laboratory, or a health care provider of the patient. The business herein or the CLIA laboratory performs one or more of the following steps: (a) flowing the sample through a size-based separation module adapted to remove red blood cells and platelets from the sample; (b) flowing the sample through a capture module that is coupled to anti-CD71 antibodies and selectively binds red blood cells over white blood cells; (c) enriching the sample using magnetic beads (e.g., coated with CD71 to repeat the enrichment step conducted before); (d) arraying the enriched cells (e.g., on a cytospin 2D slide); (e) adding to the enriched cells one or more FISH probes such as those that specifically bind the X and/or Y chromosomes; (f) using an analyzer/detection module to detect the FISH probes; (g) identify from those enriched cells nucleated red blood cells or more preferably fetal nucleated red blood cells and optionally characterize them; and (h) provide a report e.g., to the patient tested, health care provider, or insurance diagnosing a fetus with presence or absence of a fetal abnormality.

FIG. 10B illustrates another embodiment of the business methods disclosed herein. A sample of 32-40 mL of blood is drawn from a pregnant woman. The sample is flowed through an automated size-based separation module adapted to remove red blood cells and platelets from the sample. The automated separation module is coupled to a delivery apparatus. The sample is then flowed through a capture module coupled to anti-GPA antibodies. The sample is then enriched using magnetic beads (e.g., coated with CD71 to repeat the enrichment step conducted before). The remaining enriched cells are arrayed on a cytospin 2D slide with FISH probes for chromosomes X, Y, 13, 18, and 21. The FISH probes are then automatically read using an analyzer/detection module as described herein or preferably a multi-sepctral imaging system to identify and categorize nucleated RBC. Finally a report is generated for the patient tested, health care provider, or insurance diagnosing a fetus with presence or absence of a fetal abnormality.

Diagnostic Services—Oncology

In some embodiments, the business methods herein contemplate providing an oncology screening service. Fluid sample(s) (e.g., blood) from a patient to be diagnosed are obtained by the business. The business then performs one or more enrichment steps on the sample to enrich from the sample one or more cancer cells, tumor cells, epithelial cells, endothelial cells, or other cells that indicate the presence of a cancer. The above cells can be enriched from a fluid sample using any of the systems and methods disclosed herein. After enrichment, cells can be further analyzed (e.g., enumerated, assayed for specific biomarkers, etc.) to determine if the patient has or does not have cancer, original of the cancer, effective therapy for the patient, metastasis of the cancer, etc. A report generated by the business herein can be provided directly to the patient, or to a health care provider or insurance company of the patient.

Diagnostic Services—Infection

In some embodiments, the business methods herein contemplate providing an infection screening service. Such service involves obtaining a fluid sample (e.g., urine or blood) from a mammal to be diagnosed with an infection. In some embodiments, the business can draw blood or obtain the sample from the patient (animal) directly. In some embodiments, samples are delivered to the business. The business then performs a screening test on the sample to enrich from the sample one or more infected cells (e.g., infected white blood cells) or infectious organisms e.g., bacteria cells, viruses, or protozoans. The infectious organisms can be enriched by the business using the systems and methods disclosed herein. Examples of circulating pathogens that can be separated/enriched by the methods herein include, viruses (e.g., HIV, flu, SARS), bacteria (E. coli, H. influenza, S. pneumonia, M. meningitis, etc.), and protozoa (Plasmodium, Trypanosoma brucei, etc.). In some embodiments, the methods herein are used to separate and detect HIV infected cells in a blood sample. A report generated by the business herein can be provided directly to the patient, or to a health care provider or insurance company of the patient.

Diagnostic Products

In some embodiments, a business method of the present invention commercializes a diagnostic product adapted to enrich one or more analytes from a fluid sample. For example, one business method herein contemplates selling one or more of the devices/modules herein either independently or optionally in a kit with one or more reagent(s) (e.g., labeling reagents) for the separation and optional analysis of fetal cells. Such kit can include instructions for making a prenatal diagnosis. Another business method herein contemplates selling one or more of the /modules herein either independently or optionally in a kit with one or more reagent(s) (e.g., labeling reagents) for the separation and optional analysis of circulating cancer cells. Such kit can include instructions for making a cancer diagnosis. Another business method herein contemplates selling one or more of the /modules herein either independently or optionally in a kit with one or more reagent(s) (e.g., labeling reagents) for the separation and optional analysis of circulating epithelial cells. Such kit can include instructions for making a cancer diagnosis. Another business method herein contemplates selling one or more of the /modules herein either independently or optionally in a kit with one or more reagent(s) (e.g., labeling reagents) for the separation and optional analysis of circulating endothelial cells. Such kit can include instructions for making a cancer diagnosis.

In preferred embodiments, a diagnostic product comprises one or more separation module(s) and optionally one or more capture module(s). The diagnostic product can optionally include a detection/analysis tool (e.g., a computer code or software) for detecting a condition.

In some embodiments, the business method herein manufactures the diagnostic tools. In some embodiments, the business method licenses a third party to manufacture the diagnostic tools. In any of the embodiments herein, the diagnostic tool is preferably manufactured from a polymer material and is optionally disposable.

Isolation of Analytes

In some embodiments, a business method isolates one or more analytes from a sample using the systems and methods herein in exchange for a fee or a cross license. The samples can be, for example, a blood sample or other bodily sample. In some embodiments, a CLIA lab or other third party entity provides blood samples to the business to isolate rare cells such as fetal cell, epithelial cells, or cancer cells from a blood sample using the systems and methods herein. In some embodiments, the business obtains blood samples from one or more individuals and separates form such blood samples one or more therapeutic blood components such as, for example, platelets, white blood cells, circulating stem cells, etc. Such blood components can then be sold by the business for a fee. Such blood product can have a research and/or a therapeutic purpose.

VII. Manufacturing

In this example, standard photolithography is used to create a photoresist pattern of obstacles on a silicon-on-insulator (SOI) wafer. A SOI wafer consists of a 100 μm thick Si(100) layer atop a 1 μm thick SiO₂ layer on a 500 μm thick Si(100) wafer. To optimize photoresist adhesion, the SOI wafers may be exposed to high-temperature vapors of hexamethyldisilazane prior to photoresist coating. LW-sensitive photoresist is spin coated on the wafer, baked for 30 minutes at 90° C., exposed to UV light for 300 seconds through a chrome contact mask, developed for 5 minutes in developer, and post-baked for 30 minutes at 90° C. The process parameters may be altered depending on the nature and thickness of the photoresist. The pattern of the contact chrome mask is transferred to the photoresist and determines the geometry of the obstacles.

Upon the formation of the photoresist pattern that is the same as that of the obstacles, the etching is initiated. SiO₂ may serve as a stopper to the etching process. The etching may also be controlled to stop at a given depth without the use of a stopper layer. The photoresist pattern is transferred to the 100 μm thick Si layer in a plasma etcher. Multiplexed deep etching may be utilized to achieve uniform obstacles. For example, the substrate is exposed for 15 seconds to a fluorine-rich plasma flowing SF₆, and then the system is switched to a fluorocarbon-rich plasma flowing only C₄F₈ for 10 seconds, which coats all surfaces with a protective film. In the subsequent etching cycle, the exposure to ion bombardment clears the polymer preferentially from horizontal surfaces and the cycle is repeated multiple times until, e.g., the SiO₂ layer is reached.

To couple a binding moiety to the surfaces of the obstacles, the substrate may be exposed to an oxygen plasma prior to surface modification to create a silicon dioxide layer, to which binding moieties may be attached. The substrate may then be rinsed twice in distilled, deionized water and allowed to air dry. Silane immobilization onto exposed glass is performed by immersing samples for 30 seconds in freshly prepared, 2% v/v solution of 3-[(2-aminoethyl)amino] propyltrimethoxysilane in water followed by further washing in distilled, deionized water. The substrate is then dried in nitrogen gas and baked. Next, the substrate is immersed in 2.5% v/v solution of glutaraldehyde in phosphate buffered saline for 1 hour at ambient temperature. The substrate is then rinsed again, and immersed in a solution of 0.5 mg/mL binding moiety, e.g., anti-CD71, in distilled, deionized water for 15 minutes at ambient temperature to couple the binding agent to the obstacles. The substrate is then rinsed twice in distilled, deionized water, and soaked overnight in 70% ethanol for sterilization.

There are multiple techniques other than the method described above by which binding moieties may be immobilized onto the obstacles and the surfaces of the device. Simply physio-absorption onto the surface may be the choice for simplicity and cost. Another approach may use self-assembled monolayers (e.g., thiols on gold) that are functionalized with various binding moieties. Additional methods may be used depending on the binding moieties being bound and the material used to fabricate the device. Surface modification methods are known in the art. In addition, certain cells may preferentially bind to the unaltered surface of a material. For example, some cells may bind preferentially to positively charged, negatively charged, or hydrophobic surfaces or to chemical groups present in certain polymers.

The next step involves the creation of a flow device by bonding a top layer to the microfabricated silicon containing the obstacles. The top substrate may be glass to provide visual observation of cells during and after capture. Thermal bonding or a UV curable epoxy may be used to create the flow chamber. The top and bottom may also be compression fit, for example, using a silicone gasket. Such a compression fit may be reversible. Other methods of bonding (e.g., wafer bonding) are known in the art. The method employed may depend on the nature of the materials used.

The cell depletion device may be made out of different materials. Depending on the choice of the material different fabrication techniques may also be used. The device may be made out of plastic, such as polystyrene, using a hot embossing technique. The obstacles and the necessary other structures are embossed into the plastic to create the bottom surface. A top layer may then be bonded to the bottom layer. Injection molding is another approach that can be used to create such a device. Soft lithography may also be utilized to create either a whole chamber made out of poly(demethylsiloxane) (PDMS), or only the obstacles may be created in PDMS and then bonded to a glass substrate to create the closed chamber. Yet another approach involves the use of epoxy casting techniques to create the obstacles through the use of UV or temperature curable epoxy on a master that has the negative replica of the intended structure. Laser or other types of micromachining approaches may also be utilized to create the flow chamber. Other suitable polymers that may be used in the fabrication of the device are polycarbonate, polyethylene, and poly(methyl methacrylate). In addition, metals like steel and nickel may also be used to fabricate the device of the invention, e.g., by traditional metal machining. Three-dimensional fabrication techniques (e.g., stereolithography) may be employed to fabricate a device in one piece. Other methods for fabrication are known in the art.

### EXAMPLES

### Example 1

### A Silicon Device Multiplexing 14 Three-Stage Array Duplexes

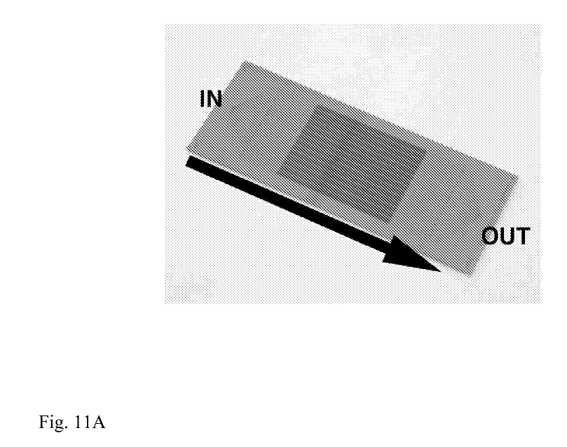

FIGS. 11A-11E show an exemplary size-based separation module of the invention, characterized as follows:

Dimensions: 90 mm×34 mm×1 mm

Array design: 3 stages, gap size=18, 12 and 8 μm for the first, second and third stage, respectively. Bifurcation ratio=1/10. Duplex; single bypass channel

Device design: multiplexing 14 array duplexes; flow resistors for flow stability

Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device packaging: The device was mechanically mated to a plastic manifold with external fluidic reservoirs to deliver blood and buffer to the device and extract the generated fractions.

Device operation: An external pressure source was used to apply a pressure of 2.4 PSI to the buffer and blood reservoirs to modulate fluidic delivery and extraction from the packaged device.

Experimental conditions: human blood from consenting adult donors was collected into K₂EDTA vacutainers (366643, Becton Dickinson, Franklin Lakes, N.J.). The undiluted blood was processed using the exemplary device described above at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.). containing 1% Bovine Serum Albumin (BSA) (A8412-100ML, Sigma-Aldrich, St Louis, Mo.).

Measurement techniques: Complete blood counts were determined using a Coulter impedance hematology analyzer (COULTER®Ac•T diff™, Beckman Coulter, Fullerton, Calif.).

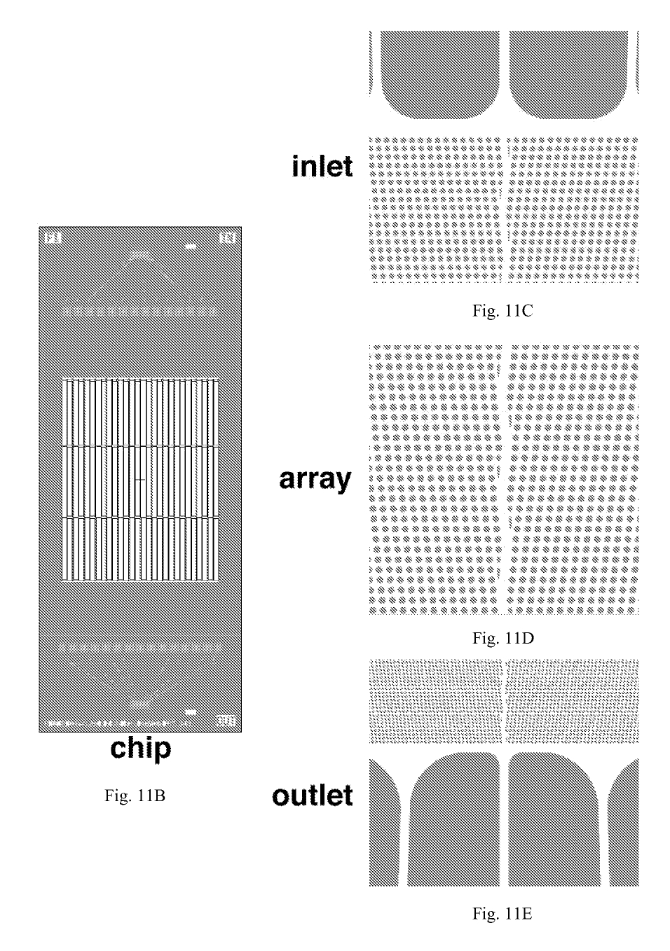

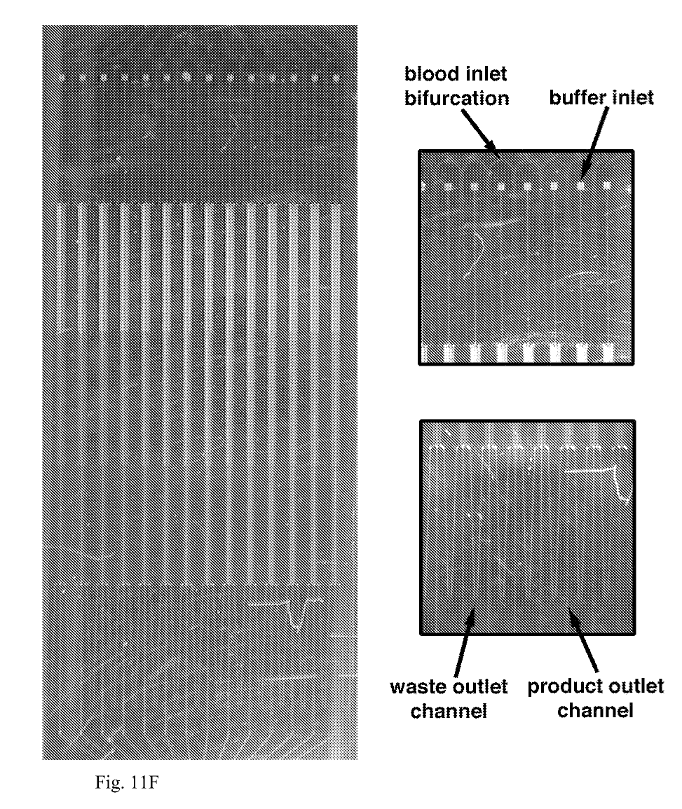

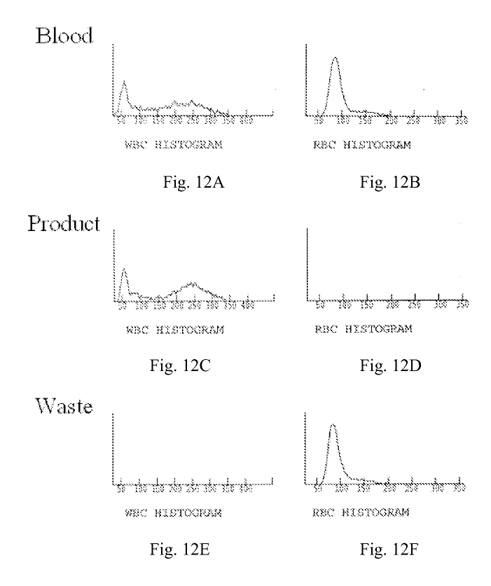

Performance: FIGS. 12A-12F shows typical histograms generated by the hematology analyzer from a blood sample and the waste (buffer, plasma, red blood cells, and platelets) and product (buffer and nucleated cells) fractions generated by the device. The following table shows the performance over 5 different blood samples:

| | | Performance Metrics | | |
|---|---|---|---|---|
| a) Sample | i) | RBC | Platelet | |
| number | Tthroughput | removal | removal | WBC loss |
| 1 | 4 mL/hr | 100% | 99% | <1% |
| 2 | 6 mL/hr | 100% | 99% | <1% |
| 3 | 6 mL/hr | 100% | 99% | <1% |
| 4 | 6 mL/hr | 100% | 97% | <1% |
| 5 | 6 mL/hr | 100% | 98% | <1% |

### Example 2

### A silicon device multiplexing 14 single-stage array duplexes

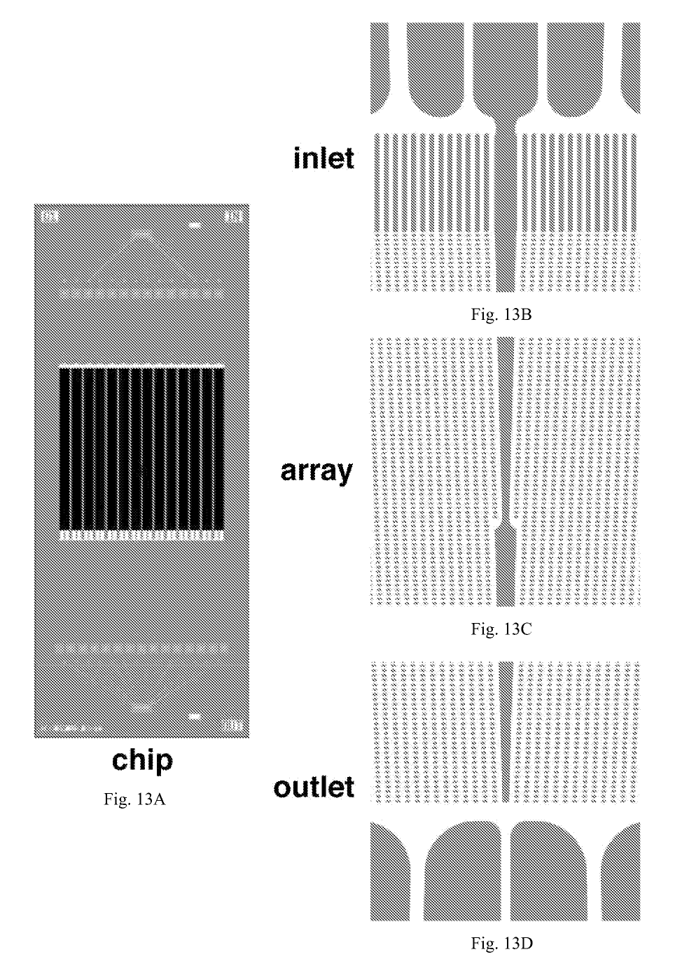

FIGS. 13A-13D shows an exemplary device of the invention, characterized as follows.

Dimensions: 90 mm×34 mm×1 mm

Array design: 1 stage, gap size=24 μm. Bifurcation ratio=1/60. Duplex; double bypass channel

Device design: multiplexing 14 array duplexes; flow resistors for flow stability

Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device packaging: The device was mechanically mated to a plastic manifold with external fluidic reservoirs to deliver blood and buffer to the device and extract the generated fractions.

Device operation: An external pressure source was used to apply a pressure of 2.4 PSI to the buffer and blood reservoirs to modulate fluidic delivery and extraction from the packaged device.

Experimental conditions: human blood from consenting adult donors was collected into K₂EDTA vacutainers (366643, Becton Dickinson, Franklin Lakes, N.J.). The undiluted blood was processed using the exemplary device described above at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.).

Measurement techniques: Complete blood counts were determined using a Coulter impedance hematology analyzer (COULTER® Ac•T difform, Beckman Coulter, Fullerton, Calif.).

Performance: The device operated at 17 mL/hr and achieved >99% red blood cell removal, >95% nucleated cell retention, and >98% platelet removal.

### Example 3

### Separation of Fetal Cord Blood

FIGS. 14A-14D shows a schematic of the device used to separate nucleated cells from fetal cord blood.

Dimensions: 100 mm×28 mm×1 mm

Array design: 3 stages, gap size=18, 12 and 8 μm for the first, second and third stage, respectively. Bifurcation ratio=1/10. Duplex; single bypass channel.

Device design: multiplexing 10 array duplexes; flow resistors for flow stability.

Device fabrication: The arrays and channels were fabricated in silicon using standard photolithography and deep silicon reactive etching techniques. The etch depth is 140 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device packaging: The device was mechanically mated to a plastic manifold with external fluidic reservoirs to deliver blood and buffer to the device and extract the generated fractions.

Device operation: An external pressure source was used to apply a pressure of 2.0 PSI to the buffer and blood reservoirs to modulate fluidic delivery and extraction from the packaged device.

Experimental conditions: Human fetal cord blood was drawn into phosphate buffered saline containing Acid Citrate Dextrose anticoagulants. 1 mL of blood was processed at 3 mL/hr using the device described above at room temperature and within 48 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.) and 2 mM EDTA (15575-020, Invitrogen, Carlsbad, Calif.).

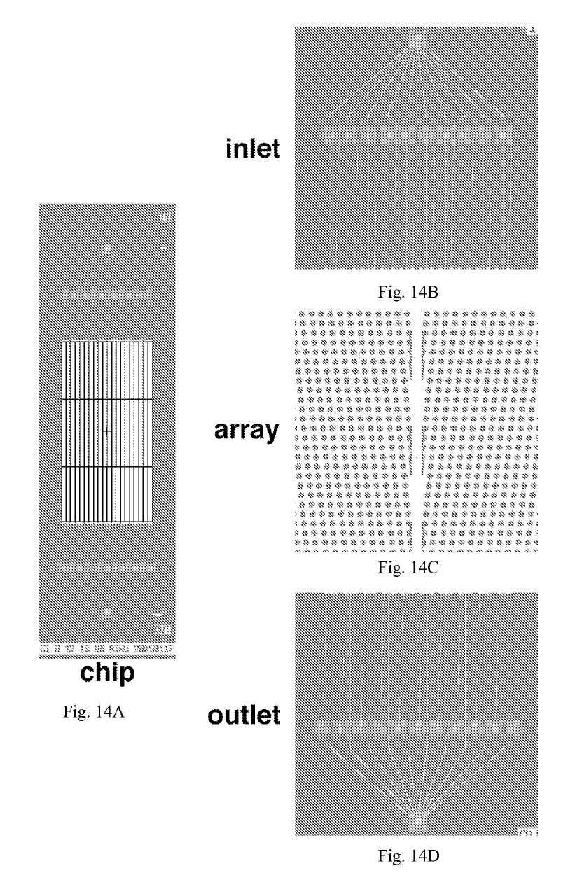

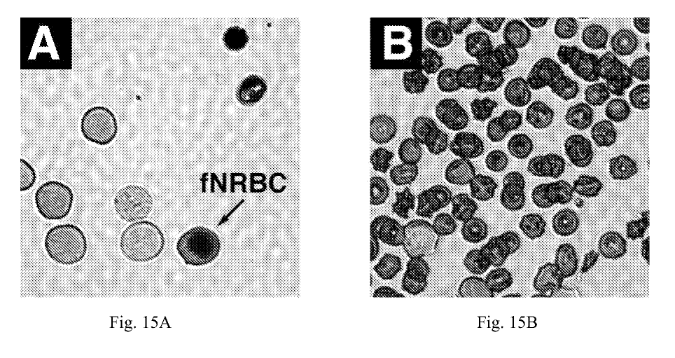

Measurement techniques: Cell smears of the product and waste fractions (FIG. 15A-15B) were prepared and stained with modified Wright-Giemsa (WG 16, Sigma Aldrich, St. Louis, Mo.).

Performance: Fetal nucleated red blood cells were observed in the product fraction (FIG. 15A) and absent from the waste fraction (FIG. 1 SB).

### Example 4

### Isolation of Fetal Cells from Maternal blood

The device and process described in detail in Example 1 were used in combination with immunomagnetic affinity enrichment techniques to demonstrate the feasibility of isolating fetal cells from maternal blood.

Experimental conditions: blood from consenting maternal donors carrying male fetuses was collected into K₂EDTA vacutainers (366643, Becton Dickinson, Franklin Lakes, N.J.) immediately following elective termination of pregnancy. The undiluted blood was processed using the device described in Example 1 at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.). Subsequently, the nucleated cell fraction was labeled with anti-CD71 microbeads (130-046-201, Miltenyi Biotech Inc., Auburn, Calif.) and enriched using the MiniMACS™ MS column (130-042-201, Miltenyi Biotech Inc., Auburn, Calif.) according to the manufacturer's specifications. Finally, the CD71-positive fraction was spotted onto glass slides.

Measurement techniques: Spotted slides were stained using fluorescence in situ hybridization (FISH) techniques according to the manufacturer's specifications using Vysis probes (Abbott Laboratories, Downer's Grove, Ill.). Samples were stained from the presence of X and Y chromosomes. In one case, a sample prepared from a known Trisomy 21 pregnancy was also stained for chromosome 21.

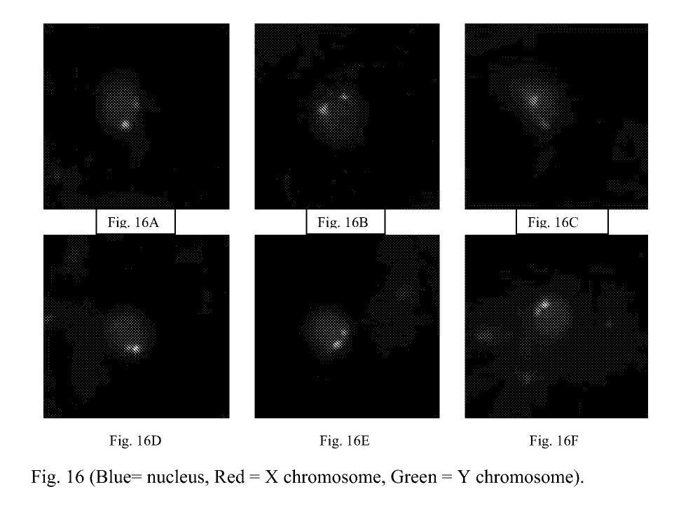

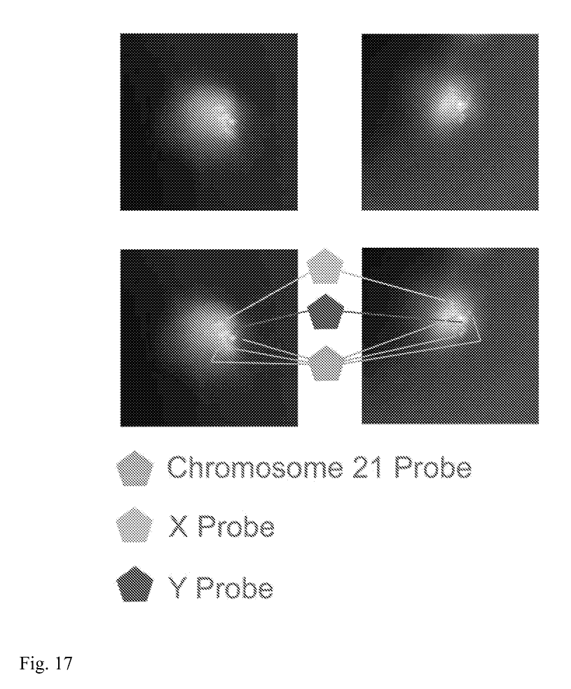

Performance: Isolation of fetal cells was confirmed by the reliable presence of male cells in the CD71-positive population prepared from the nucleated cell fractions (FIG. 16). In the single abnormal case tested, the trisomy 21 pathology was also identified (FIG. 17).

The following examples show specific embodiments of devices of the invention. The description for each device provides the number of stages in series, the gap size for each stage, E (Flow Angle), and the number of channels per device (Arrays/Chip). Each device was fabricated out of silicon using DRIE, and each device had a thermal oxide layer.

### Example 5

This device includes five stages in a single array.

| Array Design: | 5 stage, asymmetric array | |
|---|---|---|
| Gap Sizes: | Stage 1: | 8 μm |
| | Stage 2: | 10 μm |
| | Stage 3: | 12 μm |
| | Stage 4: | 14 μm |
| | Stage 5: | 16 μm |

| | |
|---|---|
| Flow Angle: | 1/10 |
| Arrays/Chip: | 1 |

### Example 6

This device includes the stages, where each stage is a duplex having a bypass channel. The height of the device was 125 μm.

| Array Design: | | symmetric 3 stage array | |
|---|---|---|---|
| | | with central collection | |
| | | channel | |
| | Gap Sizes: | Stage 1: | 8 μm |
| | | Stage 2: | 12 μm |
| | | Stage 3: | 18 μm |

| | |
|---|---|
| Flow Angle: | 1/10 |
| Arrays/Chip: | 1 |
| Other | central collection channel |

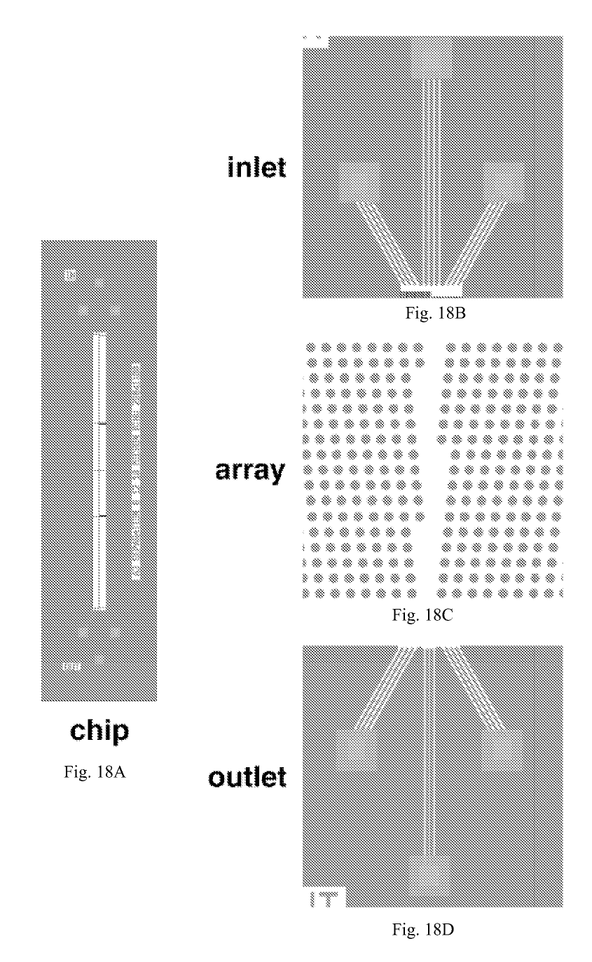

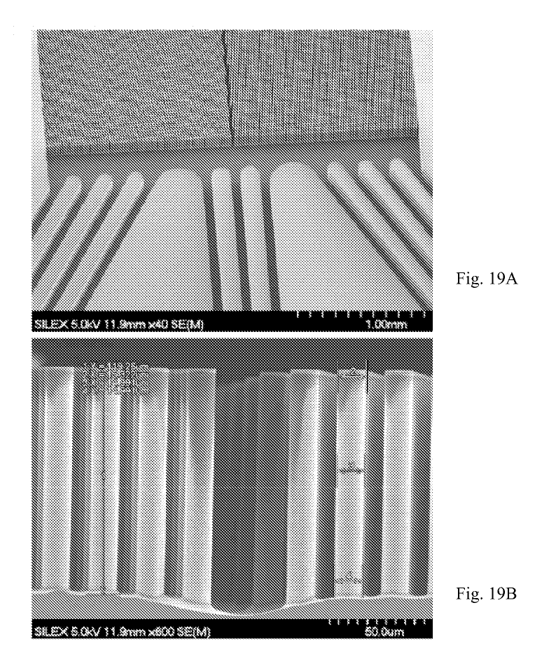

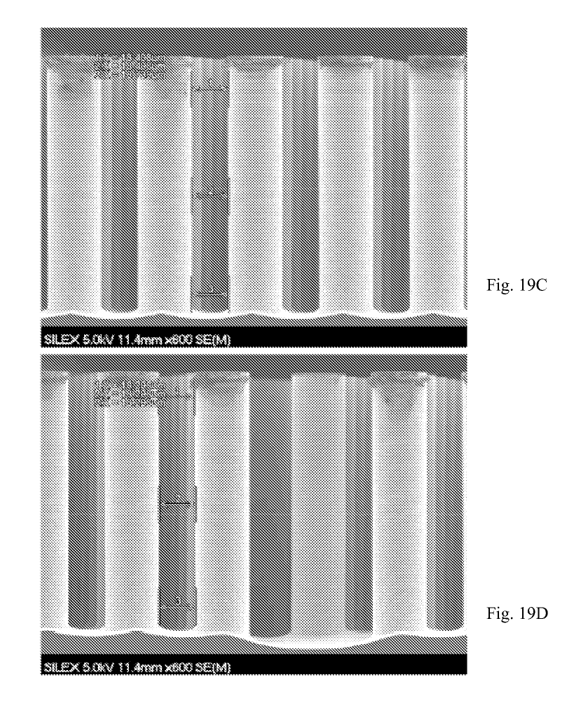

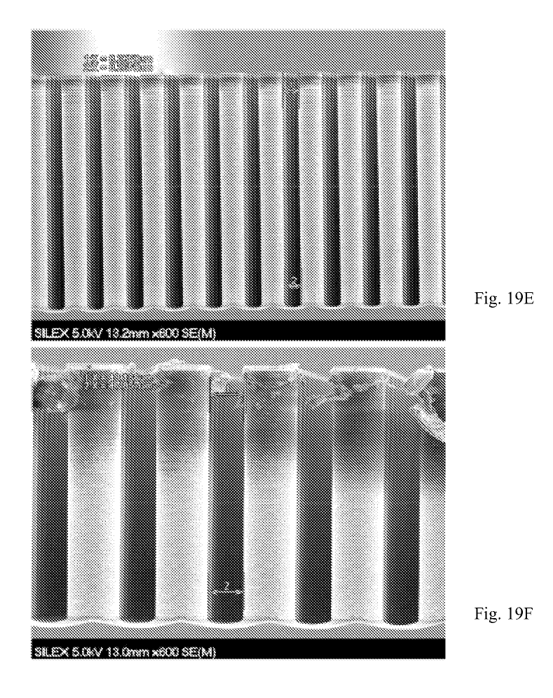

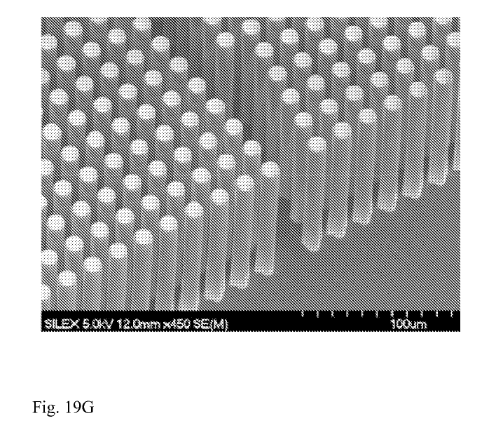

FIG. 18A shows the mask employed to fabricate a size-based separation device herein. FIGS. 18B-18D are enlargements of the portions of the mask that define the inlet, array, and outlet. FIGS. 19A-19G show SEMs of a size-based separation module herein.

### Example 7

This device includes the stages, where each stage is a duplex having a bypass channel. “Fins” were designed to flank the bypass channel to keep fluid from the bypass channel from re-entering the array. The chip also included on-chip flow resistors, i.e., the inlets and outlets possessed greater fluidic resistance than the array. The height of the device was 117 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | large fin central collection channel |
| | on-chip flow resistors |

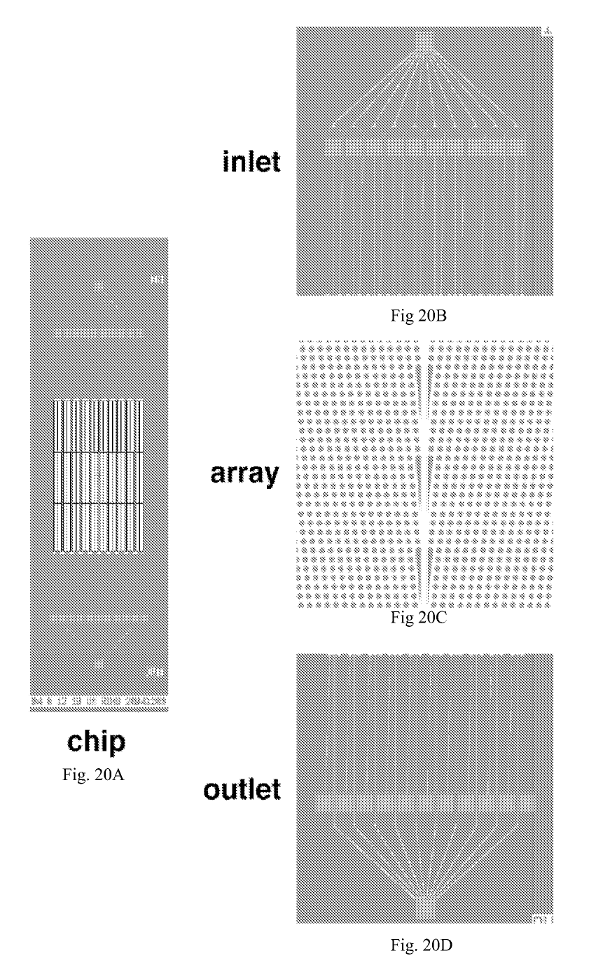

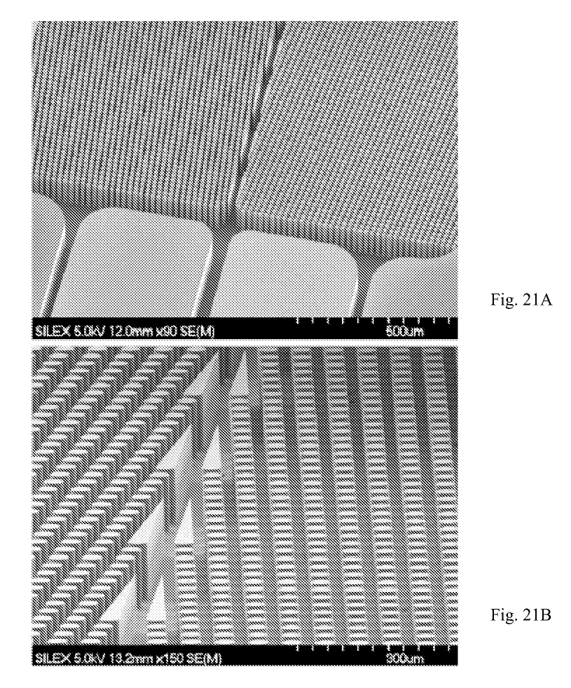

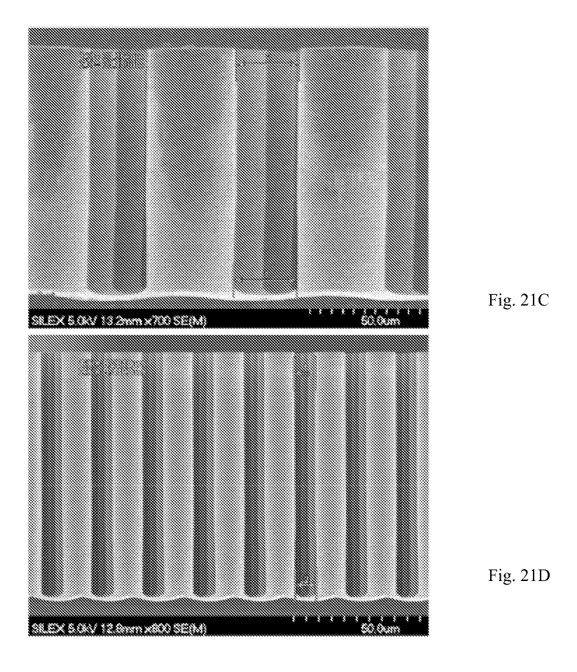

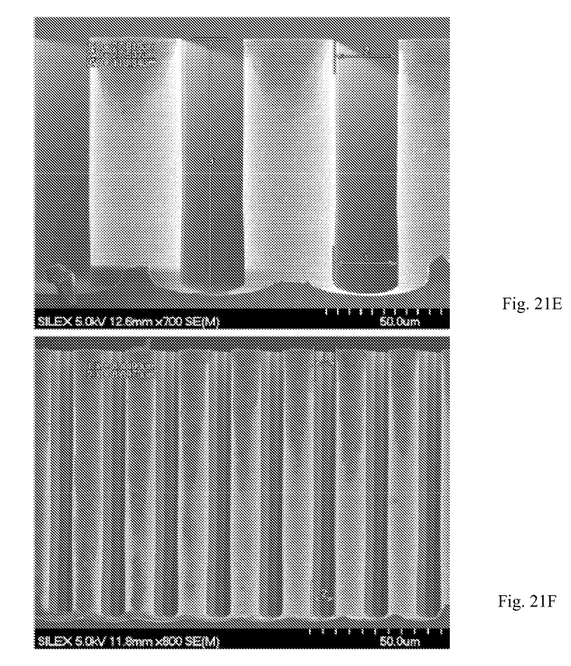

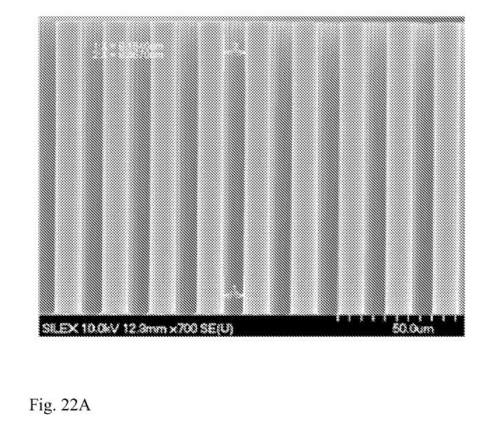

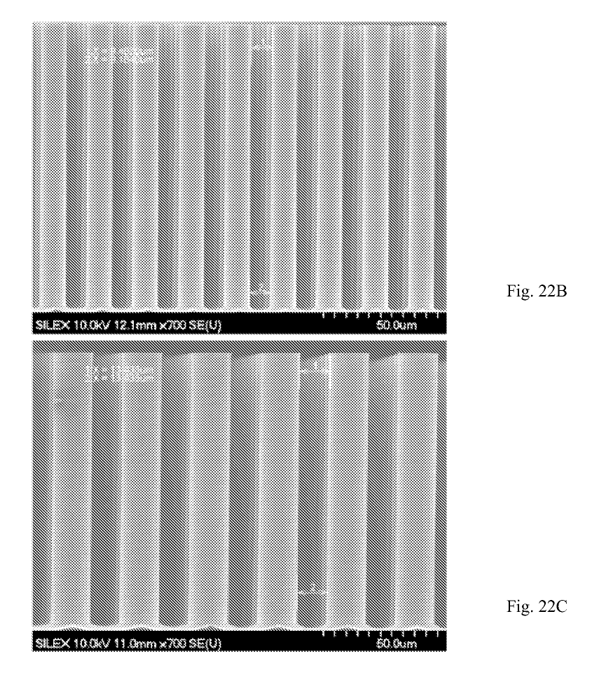

FIG. 20A shows the mask employed to fabricate a size-based separation module herein. FIGS. 20B-20D are enlargements of the portions of the mask that define the inlet, array, and outlet. FIGS. 21A-21F show SEMs of a separation module used in this example.

### Example 8

This device includes the stages, where each stage is a duplex having a bypass channel. “Fins” were designed to flank the bypass channel to keep fluid from the bypass channel from re-entering the array. The edge of the fin closest to the array was designed to mimic the shape of the array. The chip also included on-chip flow resistors, i.e., the inlets and outlets possessed greater fluidic resistance than the array. The height of the device was 138 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | alternate large fin central collection channel |
| | on-chip flow resistors |

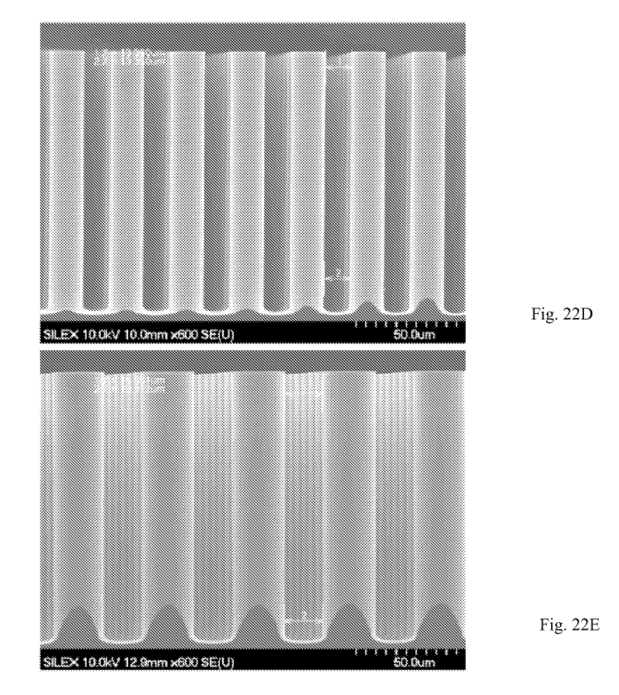

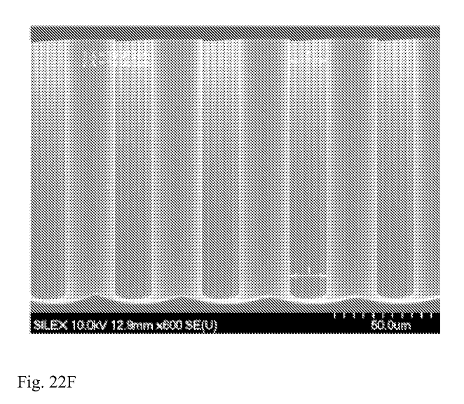

FIG. 14A shows the mask employed to fabricate the device. FIGS. 14B-14D are enlargements of the portions of the mask that define the inlet, array, and outlet. FIGS. 22A-22F show SEMs of a device as described above.

### Example 9

This device includes the stages, where each stage is a duplex having a bypass channel. “Fins” were optimized using Femlab to flank the bypass channel to keep fluid from the bypass channel from re-entering the array. The edge of the fin closest to the array was designed to mimic the shape of the array. The chip also included on-chip flow resistors, i.e., the inlets and outlets possessed greater fluidic resistance than the array. The height of the device was 139 or 142 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | Femlab optimized central collection channel (Femlab I) |
| | on-chip flow resistors |

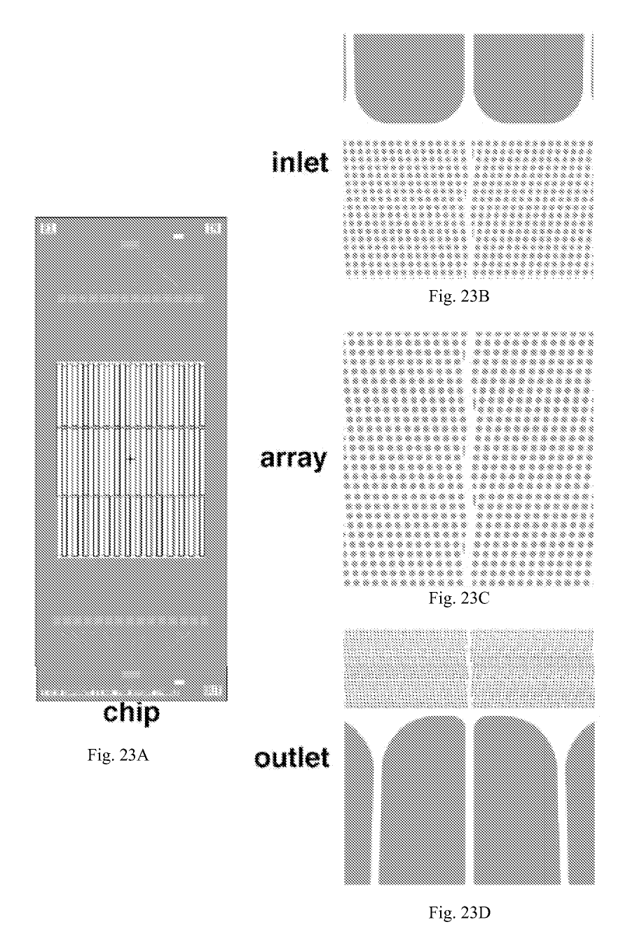

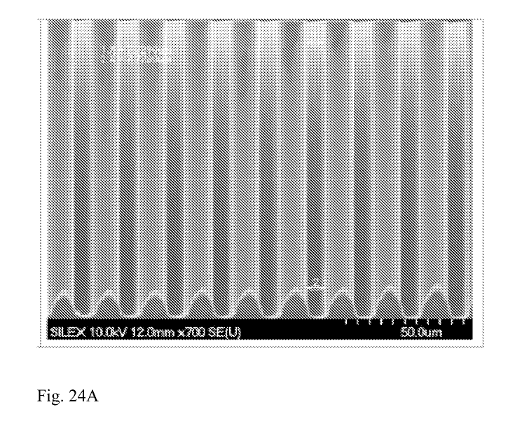

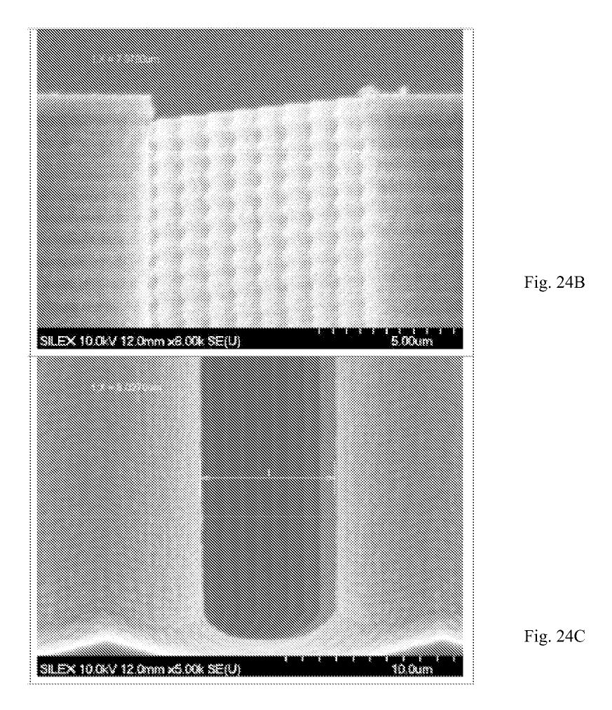

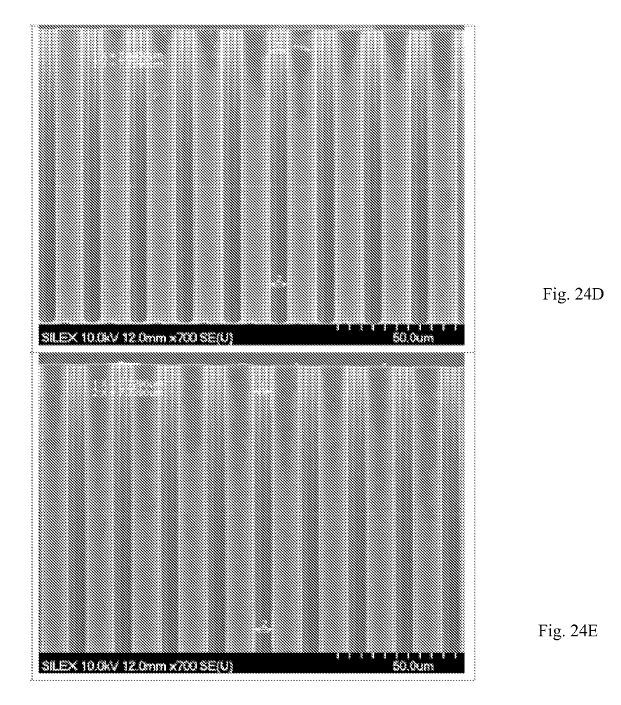

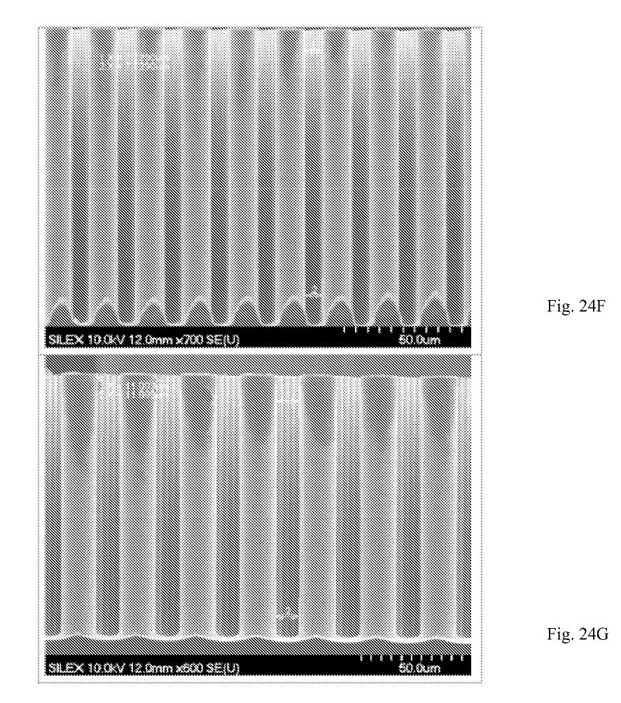

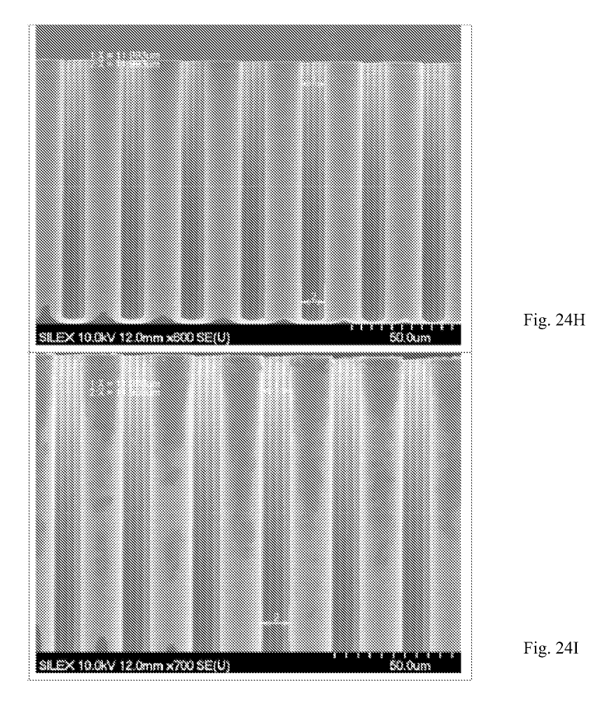

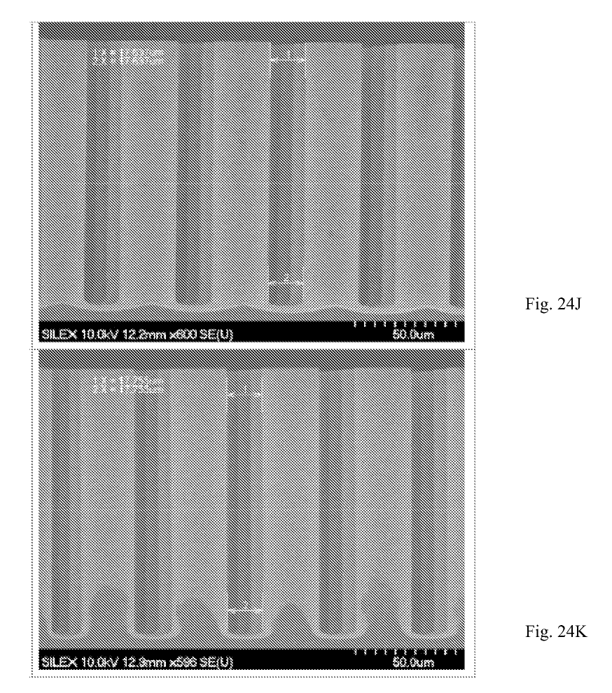

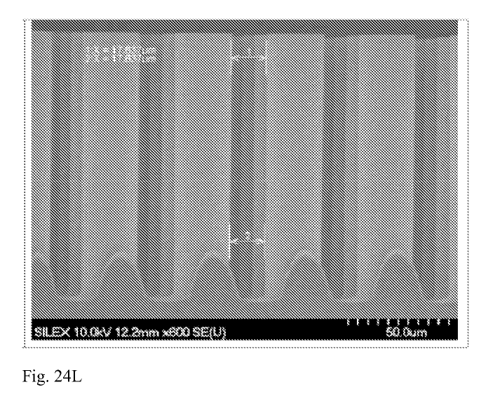

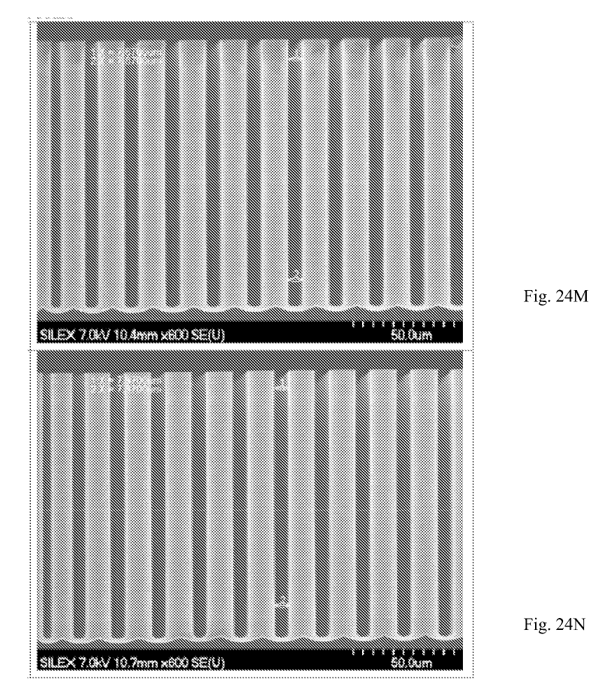

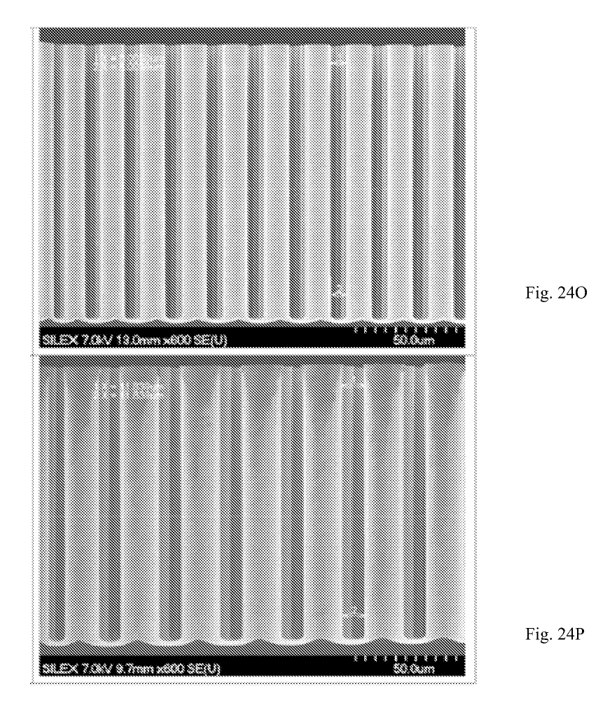

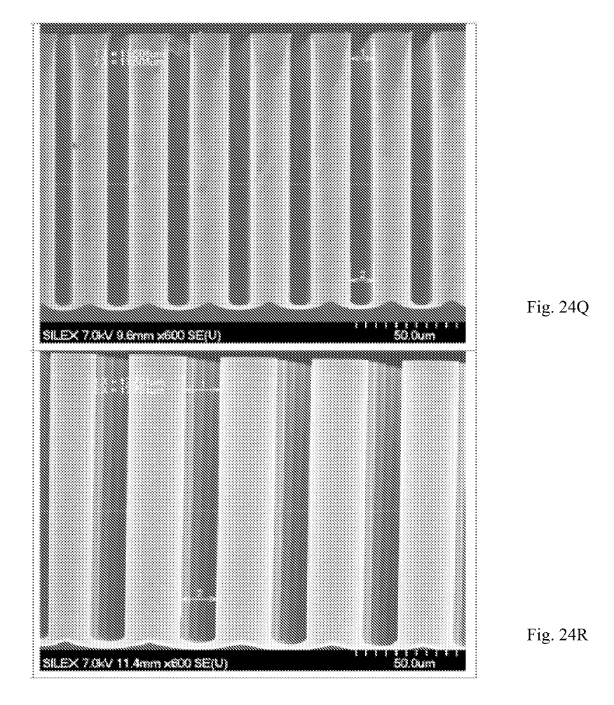

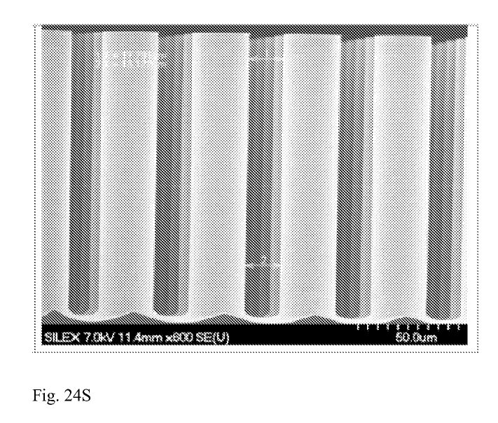

FIG. 23A shows the mask employed to fabricate the device. FIGS. 23B-23D are enlargements of the portions of the mask that define the inlet, array, and outlet. FIGS. 24A-24S show SEMs of the above device.

### Example 10

This device includes a single stage, duplex device having a bypass channel disposed to receive output from the ends of both arrays. The obstacles in this device are elliptical. The array boundary was modeled in Femlab to. The chip also included on-chip flow resistors, i.e., the inlets and outlets possessed greater fluidic resistance than the array. The height of the device was 152 μm.

| Array Design: | single stage symmetric array | |
|---|---|---|
| Gap Sizes: | Stage 1: | 24 μm |

| | |
|---|---|
| Flow Angle: | 1/60 |
| Arrays/Chip: | 14 |
| Other | central barrier |
| | ellipsoid posts |
| | on-chip resistors |
| | Femlab modeled array |
| | boundary |

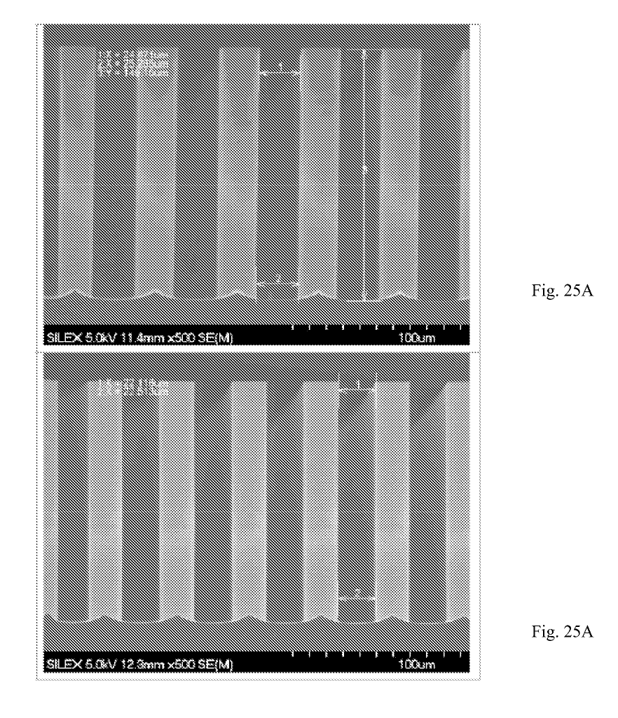

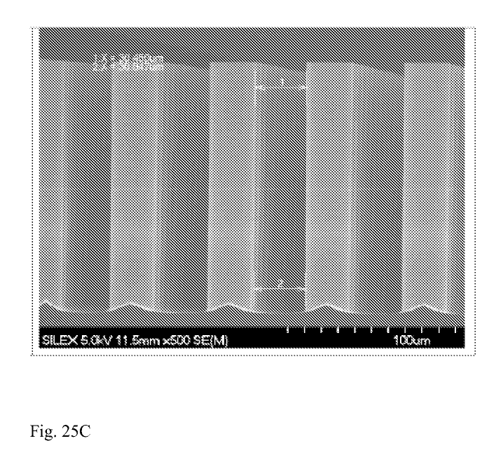

FIG. 13A shows the mask employed to fabricate the device. FIGS. 13B-13D are enlargements of the portions of the mask that define the inlet, array, and outlet. FIGS. 25A-25C show SEMs of the actual device.

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of identifying fetal abnormality, comprising:
delivering a maternal blood sample from a pregnant female to a flow though module which deterministically separates fetal cells in said sample; delivering said separated fetal cells to an analyzer adapted for capturing an image of one or more fetal cell enriched from said blood sample;
analyzing signals from one or more nucleic acid probes that bind fetal nucleic acids;
analyzing said signals; and
generating a diagnostic output according based on said analyzing step.

2. The method of claim 1 wherein said probes are specific to a chromosome.

3. The method of claim 2 wherein said chromosome is selected from the group consisting of: X chromosome, Y chromosome, chromosome 21, chromosome 13 and chromosome 18.

4. The method of claim 2 wherein said analyzing comprises determining number of said probe signals, determining size of said probe signals, determining shape of said probe signals, determining aspect ratio of said probe signals, or determining distribution of said probe signals.

5. A computer program product that detects a condition of a fetus comprising:
computer code that detects nucleated red blood cells in a sample;
computer code that receives probe signals from one or more nucleic acid probes that bind nucleic acids of interest;
computer code that analyzes said probe signals to detect one or more fetal cells;
computer code that analyzes said probe signals to detect a condition in said one or more fetal cells; and
a computer readable medium that stores the computer codes.

6. The computer program product of claim 5 wherein the computer readable medium is a memory, hard drive, floppy disk, CD-ROM, flash memory, or tape.

7. The computer program product of claim 5 wherein said probes are specific to a chromosome.

8. The computer program product of claim 7 wherein said chromosome is selected from the group consisting of: X chromosome, Y chromosome, chromosome 21, chromosome 13 and chromosome 18.

9. The computer program product of claim 5 wherein said probes are calorimetric probes.

10. The computer program product of claim 5 wherein said probes are fluorescent probes.

11. The method of claim 1 wherein said female is at 12 weeks or less gestation.

12. The method of claim 1 wherein said flow through module comprises one or more two dimensional arrays of obstacles wherein said obstacles define gaps which direct the sample flow unequally into subsequent gaps.
